(19) United States
(12) Patent Application Publication
(10) Pub. No.: US 2024/0401130 A1
(43) Pub. Date: Dec. 5, 2024

(54) SYSTEMS AND METHODS FOR SEQUENCING WITH MULTI-PRIMING

(71) Applicant: Ultima Genomics, Inc., Fremont, CA (US)

(72) Inventors: Florian OBERSTRASS, Menlo Park, CA (US); Daniel MAZUR, Newark, CA (US)

(21) Appl. No.: 18/738,596

(22) Filed: Jun. 10, 2024

Related U.S. Application Data
(63) Continuation of application No. PCT/US2022/053000, filed on Dec. 15, 2022.
(60) Provisional application No. 63/290,556, filed on Dec. 16, 2021.

(51) Int. Cl. C12Q 1/6874; C12Q 1/44; C12Q 1/6876

(52) U.S. Cl. CPC C12Q 1/6874; C12Q 1/44; C12Q 1/6876; G01N 2333/916

(57) ABSTRACT

Provided herein are systems, methods, compositions, and kits for sequencing with multi-priming. In some cases, multiple distinct primer molecules may be provided to a template nucleic acid to hybridize to distinct regions of the template nucleic acid. In some cases, a connected primer molecule may be provided to a template nucleic acid to have multiple distinct primer regions hybridize to distinct regions of the template nucleic acid. Non-adjacent regions of the template nucleic acid may be sequenced in distinct sequencing operations.

### CROSS-REFERENCE

This application is a continuation of International Application No. PCT/US2022/053000, filed Dec. 15, 2022, which claims the benefit of U.S. patent application No. 63/290,556, filed Dec. 16, 2021, which is incorporated by reference herein in its entirety.

### BACKGROUND

Biological sample processing has various applications in the fields of molecular biology and medicine (e.g., diagnosis). For example, nucleic acid sequencing may provide information that may be used to diagnose a certain condition in a subject and in some cases tailor a treatment plan. Sequencing is widely used for molecular biology applications, including vector designs, gene therapy, vaccine design, industrial strain design and verification. Biological sample processing may involve a fluidics system and/or a detection system.

Despite the advance of sequencing technology, analyzing samples with high throughput and efficiency still requires laborious efforts.

### SUMMARY

There may be multiple sequence regions of interest within a template nucleic acid. Similarly, in some cases, the template nucleic acid may have one or more sequence regions in which the sequence information is not needed or useful for a particular application. In such cases, it may be unnecessary and inefficient to obtain a single, long sequencing read that contains the multiple sequence regions. Further, a long sequencing read may have poor sequencing quality. Thus, recognized herein is a need to selectively sequence multiple, distinct regions of a template nucleic acid, and/or a need to selectively not sequence one or more distinct regions of a template nucleic acid. Provided herein are systems, methods, compositions, and kits for sequencing with multi-priming that addresses at least the abovementioned needs.

In some aspects, provided is a method, comprising: (a) hybridizing a first primer molecule and a second primer molecule to a first target region and a second target region, respectively, of a target nucleic acid strand, wherein an intermediary region is disposed between the first target region and the second target region on the target nucleic acid strand, wherein the second primer molecule is inactivated for extension; (b) extending the first primer molecule to generate a first sequencing read for a first region of the target nucleic acid strand; (c) activating the second primer molecule for extension; and (d) extending the second primer molecule to generate a second sequencing read for a second region of the target nucleic acid strand, wherein the second region is different from the first region.

In some embodiments, the target nucleic acid strand is coupled to a support. In some embodiments, the support is a bead. In some embodiments, the bead is immobilized to an individually addressable location of a plurality of individually addressable locations on a substrate. In some embodiments, the plurality of individually addressable locations comprises at least 1,000,000,000 individually addressable locations.

In some embodiments, the first target region is disposed 5′ to the second target region on the target nucleic acid strand. In some embodiments, the first target region is disposed 3′ to the second target region on the target nucleic acid strand.

In some embodiments, the first region or the second region comprises at least a portion of the intermediary region.

In some embodiments, the second primer molecule is inactivated by a blocking moiety. In some embodiments, the blocking moiety comprises a dideoxynucleotide. In some embodiments, the activating in (c) comprises cleaving the blocking moiety.

In some embodiments, the second primer molecule comprises one or more cleavage sites disposed 5′ of the blocking moiety. In some embodiments, the one or more cleavage sites comprises one or more members selected from the group consisting of: uracil, ribonucleotide, inosine, FapyG, 8oxoG, C3 spacer, photocleavable moiety, azobenzene, and abasic site. In some embodiments, the one or more cleavage sites comprises one or more uracils, and the activating in (c) comprises using a uracil-specific excision reagent (USER) enzyme to excise the one or more uracils. In some embodiments, the one or more cleavage sites comprises the photocleavable moiety, and the activating in (c) comprises applying ultraviolet radiation stimulus to the second primer molecule.

In some embodiments, the method further comprises further comprising, prior to (d), terminating extension of the first primer molecule in (b). In some embodiments, the terminating occurs prior to (c).

In some embodiments, the terminating comprises incorporation of a terminated nucleotide. In some embodiments, the terminated nucleotide comprises a dideoxynucleotide.

In some embodiments, the first primer molecule is phosphorylated, and the terminating comprises using a phosphate-dependent enzyme to degrade the first primer molecule.

In some embodiments, the method further comprises, prior to (d), degrading a first extension strand extended from the first primer molecule via an exonuclease. In some embodiments, the degrading occurs prior to (c). In some embodiments, the first primer molecule comprises a 5′-phosphate, wherein the second primer molecule does not have a 5′-phosphate, and the first extension strand is degraded using a Lambda Exonuclease. In some embodiments, a 5′ end of the second primer molecule is protected, wherein a 5′ end of the first primer molecule is unprotected, and the first extension strand is degraded using a T7 Exonuclease. In some embodiments, 3′ end of the second primer molecule is protected, and the first extension strand is degraded using an Exonuclease III.

In some embodiments, the intermediary region comprises one or more of a barcode, a sample index, and a unique molecular identifier.

In some embodiments, the intermediary region comprises between 10 and 60 bases.

In some embodiments, first target region or the second target region comprises a polyT homopolymer region. In some embodiments, the first primer molecule or the second primer molecule comprises a polyA sequence of at least 10 bases in length.

In some embodiments, the method further comprises associating the first sequencing read and the second sequencing read via a respective individually addressable location of sequencing signals detected on a substrate during generation of the first sequencing read and the second sequencing read.

In another aspect, provided is a composition, comprising: a target nucleic acid strand comprising a first target region and a second target region, wherein an intermediary region is disposed between the first target region and the second target region on the target nucleic acid strand; a first primer molecule comprising a first sequence complementary to the first target region; and a second primer molecule comprising a second sequence complementary to the second target region, wherein the second primer molecule is inactivated for extension.

In some embodiments, the composition further comprise a support coupled to the target nucleic acid strand. In some embodiments, the support is a bead. In some embodiments, the bead is immobilized to an individually addressable location of a plurality of individually addressable locations on a substrate. In some embodiments, the plurality of individually addressable locations comprises at least 1,000,000,000 individually addressable locations.

In some embodiments, the first target region is disposed 5′ to the second target region on the target nucleic acid strand. In some embodiments, the first target region is disposed 3′ to the second target region on the target nucleic acid strand.

In some embodiments, the first primer molecule or the second primer molecule is hybridized to the target nucleic acid strand. In some embodiments, the first primer molecule and the second primer molecule are hybridized to the target nucleic acid strand.

In some embodiments, the composition further comprises a growing strand extended from the first primer molecule and hybridized to the target nucleic acid strand. In some embodiments, the growing strand comprises a terminated nucleotide at a 3′ end. In some embodiments, the terminated nucleotide comprises a dideoxynucleotide.

In some embodiments, the composition further comprises an exonuclease. In some embodiments, the first primer molecule comprises a 5′-phosphate, wherein the second primer molecule does not have a 5′-phosphate, and the exonuclease is a Lambda Exonuclease. In some embodiments, a 5′ end of the second primer molecule is protected, wherein a 5′ end of the first primer molecule is unprotected, and the exonuclease is a T7 Exonuclease. In some embodiments, a 3′ end of the second primer molecule is protected, and the exonuclease is an Exonuclease III.

In some embodiments, the second primer molecule is inactivated for extension by a blocking moiety. In some embodiments, the blocking moiety comprises a dideoxynucleotide.

In some embodiments, the second primer molecule comprises one or more cleavage sites disposed 5′ of the blocking moiety. In some embodiments, the one or more cleavage sites comprises one or more members selected from the group consisting of: uracil, ribonucleotide, inosine, FapyG, 8oxoG, C3 spacer, photocleavable moiety, azobenzene, and abasic site.

In some embodiments, the one or more cleavage sites comprises one or more uracils.

In some embodiments, the one or more cleavage sites comprises the photocleavable moiety.

In some embodiments, the composition further comprises a cleavage reagent configured to cleave the second primer molecule at the one or more cleavage sites.

In some embodiments, the cleavage reagent comprises a uracil-specific excision reagent (USER).

In some embodiments, the cleavage reagent comprises a dose of ultraviolet radiation.

In some embodiments, the composition further comprises an extension termination reagent configured to terminate an extension of the first primer molecule. In some embodiments, the first primer molecule is phosphorylated, and the extension termination reagent comprises a phosphate-dependent enzyme.

In some embodiments, the intermediary region comprises one or more of a barcode, a sample index, and a unique molecular identifier.

In some embodiments, the intermediary region comprises between 10 and 60 bases.

In some embodiments, the first target region or the second target region comprises a polyT homopolymer region. In some embodiments, the first primer molecule or the second primer molecule comprises a polyA sequence of at least 10 bases in length.

In another aspect, provided is a kit, comprising: a first primer molecule comprising a first sequence complementary to a first target region; a second primer molecule comprising a second sequence complementary to a second target region, wherein the second primer molecule is inactivated for extension; and an activation reagent configured to activate the second primer molecule for extension, or instructions to activate the second primer molecule for extension.

In some embodiments, the kit further comprises a termination reagent configured to terminate an extension reaction of the first primer molecule. In some embodiments, the termination reagent comprises a dideoxynucleotide.

In some embodiments, the first primer molecule is phosphorylated, and the termination reagent comprises a phosphate-dependent enzyme.

In some embodiments, the kit further comprises a degradation reagent configured to degrade a first extension strand extended from the first primer molecule.

In some embodiments, the degradation reagent is an exonuclease. In some embodiments, the first primer molecule comprises a 5′-phosphate, wherein the second primer molecule does not have a 5′-phosphate, and the exonuclease is a Lambda Exonuclease. In some embodiments, a 5′ end of the second primer molecule is protected, wherein a 5′ end of the first primer molecule is unprotected, and the exonuclease is a T7 Exonuclease. In some embodiments, a 3′ end of the second primer molecule is protected, and the exonuclease is an Exonuclease III.

In some embodiments, the second primer molecule is inactivated for extension by a blocking moiety. In some embodiments, the blocking moiety comprises a dideoxynucleotide.

In some embodiments, the second primer molecule comprises one or more cleavage sites disposed 5′ of the blocking moiety. In some embodiments, the one or more cleavage sites comprises one or more members selected from the group consisting of uracil, ribonucleotide, inosine, FapyG, 8oxoG, C3 spacer, photocleavable moiety, azobenzene, and abasic site.

In some embodiments, the one or more cleavage sites comprises one or more uracils.

In some embodiments, the one or more cleavage sites comprises the photocleavable moiety.

In some embodiments, the activation reagent comprises a cleavage reagent configured to cleave the second primer molecule at the one or more cleavage sites. In some embodiments, the cleavage reagent comprises a uracil-specific excision reagent (USER). In some embodiments, the cleavage reagent comprises a dose of ultraviolet radiation or the instructions comprise instructions to apply the ultraviolet radiation at a predetermined wavelength.

In some embodiments, the first target region or the second target region comprises a polyT homopolymer region. In some embodiments, the first primer molecule or the second primer molecule comprises a polyA sequence of at least 10 bases in length.

In another aspect, provided is a method, comprising: (a) hybridizing a first primer region and a second primer region of a primer molecule to a first target region and a second target region, respectively, of a target nucleic acid strand, wherein an intermediary region is disposed between the first target region and the second target region on the target nucleic acid strand, wherein a connector region is disposed between the first primer region and the second primer region; (b) extending the primer molecule from the second primer region to generate a first sequencing read for a first region of the target nucleic acid strand; (c) cleaving the primer molecule to activate the first primer region for extension; and (d) extending the primer molecule from the first primer region to generate a second sequencing read for a second region of the target nucleic acid strand, wherein the second region is different from the first region.

In some embodiments, the target nucleic acid strand is coupled to a support. In some embodiments, the support is a bead. In some embodiments, the bead is immobilized to an individually addressable location of a plurality of individually addressable locations on a substrate. In some embodiments, the plurality of individually addressable locations comprises at least 1,000,000,000 individually addressable locations.

In some embodiments, the first target region is disposed 3′ to the second target region on the target nucleic acid strand.

In some embodiments, the first region or the second region comprises at least a portion of the intermediary region.

In some embodiments, the primer molecule comprises one or more cleavage sites disposed between the first primer region and the connector region. In some embodiments, the one or more cleavage sites comprises one or more members selected from the group consisting of uracil, ribonucleotide, inosine, FapyG, 8oxoG, C3 spacer, photocleavable moiety, azobenzene, and abasic site.

In some embodiments, the one or more cleavage sites comprises one or more uracils, and (c) comprises using a uracil-specific excision reagent (USER) enzyme to excise the one or more uracils.

In some embodiments, the one or more cleavage sites comprises the photocleavable moiety, and (c) comprises applying ultraviolet radiation stimulus to the primer molecule.

In some embodiments, the method further comprises, prior to (d), terminating extension of the second primer region in (b). In some embodiments, the terminating occurs prior to (c).

In some embodiments, the terminating comprises incorporation of a terminated nucleotide. In some embodiments, the terminated nucleotide comprises a dideoxynucleotide.

In some embodiments, the second primer region is phosphorylated, and the terminating comprises using a phosphate-dependent enzyme to degrade the second primer region.

In some embodiments, the method further comprises, prior to (d), degrading a first extension strand extended from the second primer region via an exonuclease. In some embodiments, a 3′ end of the first primer region is protected, and the first extension strand is degraded using an Exonuclease III.

In some embodiments, the intermediary region comprises one or more of a barcode, a sample index, and a unique molecular identifier.

In some embodiments, the intermediary region comprises between 10 and 60 bases.

In some embodiments, the first target region or the second target region comprises a polyT homopolymer region. In some embodiments, the first primer region or the second primer region comprises a polyA sequence of at least 10 bases in length.

In some embodiments, the method further comprises associating the first sequencing read and the second sequencing read via a respective individually addressable location of sequencing signals detected on a substrate during generation of the first sequencing read and the second sequencing read.

In another aspect, provided is a composition, comprising: a target nucleic acid strand comprising a first target region and a second target region, wherein an intermediary region is disposed between the first target region and the second target region on the target nucleic acid strand; and a primer molecule comprising a first primer region complementary to the first target region and a second primer region complementary to the second target region, wherein a connector region is disposed between the first primer region and the second primer region, and wherein one or more cleavage sites are disposed between the first primer region and the connector region.

In some embodiments, the composition further comprises a support coupled to the target nucleic acid strand. In some embodiments, the support is a bead. In some embodiments, the bead is immobilized to an individually addressable location of a plurality of individually addressable locations on a substrate. In some embodiments, the plurality of individually addressable locations comprises at least 1,000,000,000 individually addressable locations.

In some embodiments, the first target region is disposed 3′ to the second target region on the target nucleic acid strand.

In some embodiments, the first primer region or the second primer region is hybridized to the target nucleic acid strand. In some embodiments, the first primer region and the second primer region are hybridized to the target nucleic acid strand. In some embodiments, the composition further comprises a growing strand extended from the second primer region and hybridized to the target nucleic acid strand. In some embodiments, the growing strand comprises a terminated nucleotide at a 3′ end. In some embodiments, the terminated nucleotide comprises a dideoxynucleotide.

In some embodiments, the composition further comprises an exonuclease. In some embodiments, a 3′ end of the first primer region is protected, and the exonuclease is an Exonuclease III.

In some embodiments, the one or more cleavage sites comprises one or more members selected from the group consisting of: uracil, ribonucleotide, inosine, FapyG, 8oxoG, C3 spacer, photocleavable moiety, azobenzene, and abasic site.

In some embodiments, the one or more cleavage sites comprises one or more uracils.

In some embodiments, the one or more cleavage sites comprises the photocleavable moiety.

In some embodiments, the composition further comprises a cleavage reagent configured to cleave the primer molecule at the one or more cleavage sites. In some embodiments, the cleavage reagent comprises a uracil-specific excision reagent (USER) enzyme. In some embodiments, the cleavage reagent comprises a dose of ultraviolet radiation.

In some embodiments, the composition further comprises an extension termination reagent configured to terminate an extension of the second primer region. In some embodiments, the second primer region is phosphorylated, and the extension termination reagent comprises a phosphate-dependent enzyme. In some embodiments, the extension termination reagent comprises a dideoxynucleotide.

In some embodiments, intermediary region comprises one or more of a barcode, a sample index, and a unique molecular identifier.

In some embodiments, the intermediary region comprises between 10 and 60 bases

In some embodiments, the first target region or the second target region comprises a polyT homopolymer region. In some embodiments, the first primer region or the second primer region comprises a polyA sequence of at least 10 bases in length.

In another aspect, provided is a kit, comprising: a primer molecule comprising, in a 5′ to 3′ direction, a first primer region complementary to a first target region, a connector region, and a second primer region complementary to a second target region, wherein one or more cleavage sites are disposed between the first primer region and the connector region; and an activation reagent configured to activate the first primer region for extension, or instructions to activate the first primer region for extension.

In some embodiments, the kit further comprises a termination reagent configured to terminate an extension reaction of the second primer region. In some embodiments, the termination reagent comprises a dideoxynucleotide.

In some embodiments, the second primer region is phosphorylated, and the termination reagent comprises a phosphate-dependent enzyme.

In some embodiments, the kit further comprises a degradation reagent configured to degrade a first extension strand extended from the second primer region. In some embodiments, the degradation reagent is an exonuclease. In some embodiments, a 3′ end of the first primer region is protected, and the exonuclease is an Exonuclease III.

In some embodiments, the one or more cleavage sites comprises one or more members selected from the group consisting of: uracil, ribonucleotide, inosine, FapyG, 8oxoG, C3 spacer, photocleavable moiety, azobenzene, and abasic site.

In some embodiments, the one or more cleavage sites comprises one or more uracils.

In some embodiments, the activation reagent comprises a cleavage reagent configured to cleave the primer molecule at the one or more cleavage sites. In some embodiments, the cleavage reagent comprises a uracil-specific excision reagent (USER) enzyme. In some embodiments, the cleavage reagent comprises a dose of ultraviolet radiation or the instructions comprise instructions to apply the dose of ultraviolet radiation at a predetermined wavelength.

In some embodiments, the first target region or the second target region comprises a polyT homopolymer region. In some embodiments, the first primer region or the second primer region comprises a polyA sequence of at least 10 bases in length.

Another aspect of the present disclosure provides a system comprising one or more computer processors and computer memory coupled thereto. The computer memory comprises machine executable code that, upon execution by the one or more computer processors, implements any of the methods above or elsewhere herein. Another aspect of the present disclosure provides a non-transitory computer readable medium comprising machine executable code that, upon execution by one or more computer processors, implements any of the methods above or elsewhere herein.

Additional aspects and advantages of the present disclosure will become readily apparent to those skilled in this art from the following detailed description, wherein only illustrative instances of the present disclosure are shown and described. As will be realized, the present disclosure is capable of other and different instances, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein) of which:

FIG. 1 illustrates an example workflow for processing a sample for sequencing.

FIG. 2 illustrates examples of individually addressable locations distributed on substrates, as described herein.

FIGS. 3A-3G illustrate different examples of cross-sectional surface profiles of a substrate, as described herein.

FIG. 4 shows an example coating of a substrate with a hexagonal lattice of beads, as described herein.

FIGS. 5A-5B illustrate example systems and methods for loading a sample or a reagent onto a substrate, as described herein.

FIG. 6 illustrates a computerized system for sequencing a nucleic acid molecule.

FIGS. 7A-7C illustrate multiplexed stations in a sequencing system.

FIG. 8 illustrates a multi-priming scheme for sequencing using distinct primer molecules.

FIG. 9 illustrates an additional multi-priming scheme for sequencing using distinct primer molecules.

FIG. 10 illustrates a multi-priming scheme for sequencing using a connected primer molecule.

FIG. 11 illustrates a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the singular forms “a,” “an,” and “the” include the plural reference unless the context clearly dictates otherwise.

When a range of values is provided, it is to be understood that each intervening value between the upper and lower limit of that range, and any other stated or intervening value in that stated range is encompassed within the scope of the present disclosure. Where the stated range includes upper or lower limits, ranges excluding either of those included limits are also included in the present disclosure.

The term “biological sample,” as used herein, generally refers to any sample derived from a subject or specimen. The biological sample can be a fluid, tissue, collection of cells (e.g., cheek swab), hair sample, or feces sample. The fluid can be blood (e.g., whole blood), saliva, urine, or sweat. The tissue can be from an organ (e.g., liver, lung, or thyroid), or a mass of cellular material, such as, for example, a tumor. The biological sample can be a cellular sample or cell-free sample. Examples of biological samples include nucleic acid molecules, amino acids, polypeptides, proteins, carbohydrates, fats, or viruses. In an example, a biological sample is a nucleic acid sample including one or more nucleic acid molecules, such as deoxyribonucleic acid (DNA) and/or ribonucleic acid (RNA). The nucleic acid sample may comprise cell-free nucleic acid molecules, such as cell-free DNA or cell-free RNA. Further, samples may be extracted from variety of animal fluids containing cell free sequences, including but not limited to blood, serum, plasma, vitreous, sputum, urine, tears, perspiration, saliva, semen, mucosal excretions, mucus, spinal fluid, amniotic fluid, lymph fluid and the like. Cell free polynucleotides may be fetal in origin (via fluid taken from a pregnant subject) or may be derived from tissue of the subject itself. A biological sample may also refer to a sample engineered to mimic one or more properties (e.g., nucleic acid sequence properties, e.g., sequence identity, length, GC content, etc.) of a native sample derived from a subject or specimen.

The term “subject,” as used herein, generally refers to an individual from whom a biological sample is obtained. The subject may be a mammal or non-mammal. The subject may be human, non-human mammal, animal, ape, monkey, chimpanzee, reptilian, amphibian, avian, or a plant. The subject may be a patient. The subject may be displaying a symptom of a disease. The subject may be asymptomatic. The subject may be undergoing treatment. The subject may not be undergoing treatment. The subject can have or be suspected of having a disease, such as cancer (e.g., breast cancer, colorectal cancer, brain cancer, leukemia, lung cancer, skin cancer, liver cancer, pancreatic cancer, lymphoma, esophageal cancer, cervical cancer, etc.) or an infectious disease. The subject can have or be suspected of having a genetic disorder such as achondroplasia, alpha-1 antitrypsin deficiency, antiphospholipid syndrome, autism, autosomal dominant polycystic kidney disease, Charcot-Marie-tooth, cri du chat, Crohn's disease, cystic fibrosis, Dercum disease, down syndrome, Duane syndrome, Duchenne muscular dystrophy, factor V Leiden thrombophilia, familial hypercholesterolemia, familial Mediterranean fever, fragile x syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency, sickle cell disease, spinal muscular atrophy, Tay-Sachs, thalassemia, trimethylaminuria, Turner syndrome, velocardiofacial syndrome, WAGR syndrome, or Wilson disease.

The term “analyte,” as used herein, generally refers to an object that is the subject of analysis, or an object, regardless of being the subject of analysis, that is directly or indirectly analyzed during a process. An analyte may be synthetic. An analyte may be, originate from, and/or be derived from, a sample, such as a biological sample. In some examples, an analyte is or includes a molecule, macromolecule (e.g., nucleic acid, carbohydrate, protein, lipid, etc.), nucleic acid, carbohydrate, lipid, antibody, antibody fragment, antigen, peptide, polypeptide, protein, macromolecular group (e.g., glycoproteins, proteoglycans, ribozymes, liposomes, etc.), cell, tissue, biological particle, or an organism, or any engineered copy or variant thereof, or any combination thereof. The term “processing an analyte,” as used herein, generally refers to one or more stages of interaction with one more samples. Processing an analyte may comprise conducting a chemical reaction, biochemical reaction, enzymatic reaction, hybridization reaction, polymerization reaction, physical reaction, any other reaction, or a combination thereof with, in the presence of, or on, the analyte. Processing an analyte may comprise physical and/or chemical manipulation of the analyte. For example, processing an analyte may comprise detection of a chemical change or physical change, addition of or subtraction of material, atoms, or molecules, molecular confirmation, detection of the presence of a fluorescent label, detection of a Forster resonance energy transfer (FRET) interaction, or inference of absence of fluorescence.

The terms “nucleic acid,” “nucleic acid molecule,” “nucleic acid sequence,” “nucleic acid fragment,” “oligonucleotide” and “polynucleotide,” as used herein, generally refer to a polynucleotide that may have various lengths of bases, comprising, for example, deoxyribonucleotide, deoxyribonucleic acid (DNA), ribonucleotide, or ribonucleic acid (RNA), or analogs thereof. A nucleic acid may be single-stranded. A nucleic acid may be double-stranded. A nucleic acid may be partially double-stranded, such as to have at least one double-stranded region and at least one single-stranded region. A partially double-stranded nucleic acid may have one or more overhanging regions. An “overhang,” as used herein, generally refers to a single-stranded portion of a nucleic acid that extends from or is contiguous with a double-stranded portion of a same nucleic acid molecule. Non-limiting examples of nucleic acids include DNA, RNA, genomic DNA or synthetic DNA/RNA or coding or non-coding regions of a gene or gene fragment, loci (locus) defined from linkage analysis, exons, introns, messenger RNA (mRNA), transfer RNA, ribosomal RNA (rRNA), short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, and isolated RNA of any sequence. A nucleic acid can have a length of at least about 10 nucleic acid bases (“bases”), 20 bases, 30 bases, 40 bases, 50 bases, 100 bases, 200 bases, 300 bases, 400 bases, 500 bases, 1 kilobase (kb), 2 kb, 3, kb, 4 kb, 5 kb, 10 kb, 20 kb, 30 kb, 40 kb, 50 kb, 100 kb, 200 kb, 300 kb, 400 kb, 500 kb, 1 megabase (Mb), 10 Mb, 100 Mb, 1 gigabase or more. A nucleic acid may comprise A nucleic acid can comprise a sequence of four natural nucleotide bases: adenine (A); cytosine (C); guanine (G); and thymine (T) (uracil (U) for thymine (T) when the nucleic acid is RNA). A nucleic acid may include one or more nonstandard nucleotide(s), nucleotide analog(s) and/or modified nucleotide(s).

The term “nucleotide,” as used herein, generally refers to any nucleotide or nucleotide analog. The nucleotide may be naturally occurring or non-naturally occurring. The nucleotide may be a modified, synthesized, or engineered nucleotide. The nucleotide may include a canonical base or a non-canonical base. The nucleotide may comprise an alternative base. The nucleotide may include a modified polyphosphate chain (e.g., triphosphate coupled to a fluorophore). The nucleotide may comprise a label. The nucleotide may be terminated (e.g., reversibly terminated). Nonstandard nucleotides, nucleotide analogs, and/or modified analogs may include, but are not limited to, diaminopurine, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxy acetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxy acetic acid(v), 5-methyl-2-thiouracil, 3-(3-amino-3-N-2-carboxypropyl) uracil, (acp3)w, 2,6-diaminopurine, ethynyl nucleotide bases, 1-propynyl nucleotide bases, azido nucleotide bases, phosphoroselenoate nucleic acids and the like. In some cases, nucleotides may include modifications in their phosphate moieties, including modifications to a triphosphate moiety. Additional, non-limiting examples of modifications include phosphate chains of greater length (e.g., a phosphate chain having, 4, 5, 6, 7, 8, 9, 10 or more phosphate moieties), modifications with thiol moieties (e.g., alpha-thio triphosphate and beta-thiotriphosphates) or modifications with selenium moieties (e.g., phosphoroselenoate nucleic acids). Nucleic acids may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acids may also contain amine-modified groups, such as aminoallyl-dUTP (aa-dUTP) and aminohexhylacrylamide-dCTP (aha-dCTP) to allow covalent attachment of amine reactive moieties, such as N-hydroxysuccinimide esters (NHS). Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Nucleotides may be capable of reacting or bonding with detectable moieties for nucleotide detection.

The term “terminator” as used herein with respect to a nucleotide may generally refer to a moiety that is capable of terminating primer extension. A terminator may be a reversible terminator. A reversible terminator may comprise a blocking or capping group that is attached to the 3′-oxygen atom of a sugar moiety (e.g., a pentose) of a nucleotide or nucleotide analog. Such moieties are referred to as 3′-O-blocked reversible terminators. Examples of 3′-O-blocked reversible terminators include, for example, 3′-ONH₂ reversible terminators, 3′-O-allyl reversible terminators, and 3′-O-azidomethyl reversible terminators. Alternatively, a reversible terminator may comprise a blocking group in a linker (e.g., a cleavable linker) and/or dye moiety of a nucleotide analog. 3-unblocked reversible terminators may be attached to both the base of the nucleotide analog as well as a fluorescing group (e.g., label, as described herein). Examples of 3-unblocked reversible terminators include, for example, the “virtual terminator” developed by Helicos BioSciences Corp. and the “lightning terminator” developed by Michael L. Metzker et al. Cleavage of a reversible terminator may be achieved by, for example, irradiating a nucleic acid molecule including the reversible terminator.

The term “sequencing,” as used herein, generally refers to a process for generating or identifying a sequence of a biological molecule, such as a nucleic acid. The sequence may be a nucleic acid sequence which comprises a sequence of nucleic acid bases. As used herein, the term “template nucleic acid” generally refers to the nucleic acid to be sequenced. The template nucleic acid may be an analyte or be associated with an analyte. For example, the analyte can be a mRNA, and the template nucleic acid is the mRNA, or a cDNA derived from the mRNA, or another derivative thereof. In another example, the analyte can be a protein, and the template nucleic acid is an oligonucleotide that is conjugated to an antibody that binds to the protein, or derivative thereof. Sequencing may be single molecule sequencing or sequencing by synthesis, for example. Sequencing may comprise generating sequencing signals and/or sequencing reads. Sequencing may be performed on template nucleic acids immobilized on a support, such as a flow cell, substrate, and/or one or more beads. In some cases, a template nucleic acid may be amplified to produce a colony of nucleic acid molecules attached to the support to produce amplified sequencing signals. In one example, (i) a template nucleic acid is subjected to a nucleic acid reaction, e.g., amplification, to produce a clonal population of the nucleic acid attached to a bead, the bead immobilized to a substrate, (ii) amplified sequencing signals from the immobilized bead are detected from the substrate surface during or following one or more nucleotide flows, and (iii) the sequencing signals are processed to generate sequencing reads. The substrate surface may immobilize multiple beads at distinct locations, each bead containing distinct colonies of nucleic acids, and upon detecting the substrate surface, multiple sequencing signals may be simultaneously or substantially simultaneously processed from the different immobilized beads at the distinct locations to generate multiple sequencing reads. In some sequencing methods, the nucleotide flows comprise non-terminated nucleotides. In some sequencing methods, the nucleotide flows comprise terminated nucleotides.

The term “nucleotide flow” as used herein, generally refers to a temporally distinct instance of providing a nucleotide-containing reagent to a sequencing reaction space. The term “flow” as used herein, when not qualified by another reagent, generally refers to a nucleotide flow. For example, providing two flows may refer to (i) providing a nucleotide-containing reagent (e.g., A base-containing solution) to a sequencing reaction space at a first time point and (ii) providing a nucleotide-containing reagent (e.g., G-base containing solution) to a sequencing reaction space at a second time point different from the first time point. A “sequencing reaction space” may be any reaction environment comprising a template nucleic acid. For example, the sequencing reaction space may be or comprise a substrate surface comprising a template nucleic acid immobilized thereto; a substrate surface comprising a bead immobilized thereto, the bead comprising a template nucleic acid immobilized thereto; or any reaction chamber or surface that comprises a template nucleic acid, which may or may not be immobilized. A nucleotide flow can have any number of canonical base types (A, T, G, C; or U), for example 1, 2, 3, or 4 canonical base types. A “flow order,” as used herein, generally refers to the order of nucleotide flows used to sequence a template nucleic acid. A flow order may be expressed as a one-dimensional matrix or linear array of bases corresponding to the identities of, and arranged in chronological order of, the nucleotide flows provided to the sequencing reaction space:

(e.g., [A T G C A T G C A T G A T G A T G A T G C A T G C]).

Such one-dimensional matrix or linear array of bases in the flow order may also be referred to herein as a “flow space.” A flow order may have any number of nucleotide flows. A “flow position,” as used herein, generally refers to the sequential position of a given nucleotide flow in the flow space. A “flow cycle,” as used herein, generally refers to the order of nucleotide flow(s) of a sub-group of contiguous nucleotide flow(s) within the flow order. A flow cycle may be expressed as a one-dimensional matrix or linear array of an order of bases corresponding to the identities of, and arranged in chronological order of, the nucleotide flows provided within the sub-group of contiguous flow(s) (e.g., [A T G C], [A A T T G G C C], [A T], [A/T A/G], [A A], [A], [A T G], etc.). A flow cycle may have any number of nucleotide flows. A given flow cycle may be repeated one or more times in the flow cycle, consecutively or non-consecutively. Accordingly, the term “flow cycle order,” as used herein, generally refers to an order of flow cycles within the flow order, and can be expressed in units of flow cycles. For example, where [A T G C] is identified as a 1^{st} flow cycle, and [A T G] is identified as a 2^{nd} flow cycle, the flow order of [A T G C A T G C A T G A T G A T G A T G C A T G C] may be described as having a flow-cycle order of [1^{st} flow cycle; 1^{st} flow cycle; 2^{nd} flow cycle; 2^{nd} flow cycle; 2^{nd} flow cycle; 1^{st} flow cycle; 1^{st} flow cycle].

The terms “amplifying,” “amplification,” and “nucleic acid amplification” are used interchangeably and generally refer to generating one or more copies of a nucleic acid or a template. For example, “amplification” of DNA generally refers to generating one or more copies of a DNA molecule. Amplification of a nucleic acid may be linear, exponential, or a combination thereof. Amplification may be emulsion based or non-emulsion based. Non-limiting examples of nucleic acid amplification methods include reverse transcription, primer extension, polymerase chain reaction (PCR), ligase chain reaction (LCR), helicase-dependent amplification, asymmetric amplification, rolling circle amplification (RCA), recombinase polymerase reaction (RPA), loop mediated isothermal amplification (LAMP), nucleic acid sequence based amplification (NASBA), self-sustained sequence replication (3SR), and multiple displacement amplification (MDA). Where PCR is used, any form of PCR may be used, with non-limiting examples that include real-time PCR, allele-specific PCR, assembly PCR, asymmetric PCR, digital PCR, emulsion PCR (ePCR or emPCR), dial-out PCR, helicase-dependent PCR, nested PCR, hot start PCR, inverse PCR, methylation-specific PCR, miniprimer PCR, multiplex PCR, nested PCR, overlap-extension PCR, thermal asymmetric interlaced PCR, and touchdown PCR. Amplification can be conducted in a reaction mixture comprising various components (e.g., a primer(s), template, nucleotides, a polymerase, buffer components, co-factors, etc.) that participate or facilitate amplification. In some cases, the reaction mixture comprises a buffer that permits context independent incorporation of nucleotides. Non-limiting examples include magnesium-ion, manganese-ion and isocitrate buffers. Additional examples of such buffers are described in Tabor, S. et al. C. C. PNAS, 1989, 86, 4076-4080 and U.S. Pat. Nos. 5,409,811 and 5,674,716, each of which is herein incorporated by reference in its entirety. Useful methods for clonal amplification from single molecules include rolling circle amplification (RCA) (Lizardi et al., Nat. Genet. 19:225-232 (1998), which is incorporated herein by reference), bridge PCR (Adams and Kron, Method for Performing Amplification of Nucleic Acid with Two Primers Bound to a Single Solid Support, Mosaic Technologies, Inc. (Winter Hill, Mass.); Whitehead Institute for Biomedical Research, Cambridge, Mass., (1997); Adessi et al., Nucl. Acids Res. 28:E87 (2000); Pemov et al., Nucl. Acids Res. 33:e11(2005); or U.S. Pat. No. 5,641,658, each of which is incorporated herein by reference), polony generation (Mitra et al., Proc. Natl. Acad. Sci. USA 100:5926-5931 (2003); Mitra et al., Anal. Biochem. 320:55-65(2003), each of which is incorporated herein by reference), and clonal amplification on beads using emulsions (Dressman et al., Proc. Natl. Acad. Sci. USA 100:8817-8822 (2003), which is incorporated herein by reference) or ligation to bead-based adapter libraries (Brenner et al., Nat. Biotechnol. 18:630-634 (2000); Brenner et al., Proc. Natl. Acad. Sci. USA 97:1665-1670 (2000)); Reinartz, et al., Brief Funct. Genomic Proteomic 1:95-104 (2002), each of which is incorporated herein by reference). Amplification products from a nucleic acid may be identical or substantially identical. A nucleic acid colony resulting from amplification may have identical or substantially identical sequences.

As used herein, the terms “identical” or “percent identity,” when used with respect to two or more nucleic acid or polypeptide sequences, refer to two or more sequences that are the same or, alternatively, have a specified percentage of amino acid residues or nucleotides that are the same, when compared and aligned for maximum correspondence, as measured using any one or more of the following sequence comparison algorithms: Needleman-Wunsch (see, e.g., Needleman, Saul B.; and Wunsch, Christian D. (1970). “A general method applicable to the search for similarities in the amino acid sequence of two proteins” Journal of Molecular Biology 48 (3):443-53); Smith-Waterman (see, e.g., Smith, Temple F.; and Waterman, Michael S., “Identification of Common Molecular Subsequences” (1981) Journal of Molecular Biology 147:195-197); or BLAST (Basic Local Alignment Search Tool; see, e.g., Altschul S F, Gish W, Miller W, Myers E W, Lipman D J, “Basic local alignment search tool” (1990) J Mol Biol 215 (3):403-410). As used herein, the terms “substantially identical” or “substantial identity” when used with respect to two or more nucleic acid or polypeptide sequences, refer to two or more sequences or subsequences (such as biologically active fragments) that have at least 60%, at least 70%, at least 75%, at least 80%, at least 85%, at least 90%, at least 95%, at least 96%, at least 97%, at least 98%, or at least 99% nucleotide or amino acid residue identity, when compared and aligned for maximum correspondence, as measured using a sequence comparison algorithm or by visual inspection. Substantially identical sequences are typically considered to be homologous without reference to actual ancestry. In some embodiments, “substantial identity” exists over a region of the sequences being compared. In some embodiments, substantial identity exists over a region of at least 25 residues in length, at least 50 residues in length, at least 100 residues in length, at least 150 residues in length, at least 200 residues in length, or greater than 200 residues in length. In some embodiments, the sequences being compared are substantially identical over the full length of the sequences being compared. Typically, substantially identical nucleic acid or protein sequences include less than 100% nucleotide or amino acid residue identity as such sequences would generally be considered “identical.”

The term “coupled to,” as used herein, generally refers to an association between two or more objects that may be temporary or substantially permanent. A first object may be reversibly or irreversibly coupled to a second object. For example, a nucleic acid molecule may be reversibly coupled to a particle. A reversible coupling may comprise, for example, a releasable coupling (e.g., in which a first object may be released from a second object to which it is coupled). A first object releasably coupled to a second object may be separated from the second object, e.g., upon application of a stimulus, which stimulus may comprise a photostimulus (e.g., ultraviolet light), a thermal stimulus, a chemical stimulus (e.g., reducing agent), or any other useful stimulus. Coupling may encompass immobilization to a support (e.g., as described herein). Similarly, coupling may encompass attachment, such as attachment of a first object to a second object. Coupling may comprise any interaction that affects an association between two objects, including, for example, a covalent bond, a non-covalent interaction (e.g., electrostatic interaction [e.g., hydrogen bonding, ionic interaction, and halogen bonding], π-interaction [e.g., π-π interaction, polar-π interaction, cation-π interaction, and anion-π interaction], van der Waals force-based interactions [e.g., dipole-dipole interactions, dipole-induced dipole interactions, and induced dipole-induced dipole interactions], hydrophobic interaction), a magnetic interaction (e.g., magnetic dipole-dipole interaction, indirect dipole-dipole coupling), an electromagnetic interaction, adsorption, or any other useful interaction. For example, a particle may be coupled to a planar support via an electrostatic interaction, a magnetic interaction, or a covalent interaction. Similarly, a nucleic acid molecule may be coupled to a particle via a covalent interaction or a via a non-covalent interaction. A coupling between a first object and a second object may comprise a labile moiety, such as a moiety comprising an ester, vicinal diol, phosphodiester, peptide, glycosidic, sulfone, Diels-Alder, or similar linkage. The strength of a coupling between a first object and a second object may be indicated by a dissociation constant, K_{d}, that indicates the inclination of a coupled object comprising a first object and a second object to dissociate into the uncoupled first and second objects and may be expressed as a ratio of dissociated (e.g., uncoupled) objects to coupled objects.

The term “detector,” as used herein, generally refers to a device that is capable of detecting a signal, including a signal indicative of the presence or absence of one or more incorporated nucleotides or fluorescent labels. The detector may simultaneously or substantially simultaneously detect multiple signals. The detector may detect the signal in real-time during, substantially during a biological reaction, such as a sequencing reaction (e.g., sequencing during a primer extension reaction), or subsequent to a biological reaction. In some cases, a detector can include optical and/or electronic components that can detect signals. Non-limiting examples of detection methods, for which a detector is used, include optical detection, spectroscopic detection, electrostatic detection, electrochemical detection, acoustic detection, magnetic detection, and the like. Optical detection methods include, but are not limited to, light absorption, ultraviolet-visible (UV-vis) light absorption, infrared light absorption, light scattering, Rayleigh scattering, Raman scattering, surface-enhanced Raman scattering, Mie scattering, fluorescence, luminescence, and phosphorescence. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis. Electrochemical detection methods include, but are not limited to, electrochemical detection of amplified product after high-performance liquid chromatography separation of the amplified products. A detector may be a continuous area scanning detector. For example, the detector may comprise an imaging array sensor capable of continuous integration over a scanning area where the scanning is electronically synchronized to the image of an object in relative motion. A continuous area scanning detector may comprise a time delay and integration (TDI) charge coupled device (CCD), Hybrid TDI, complementary metal oxide semiconductor (CMOS) pseudo TDI device, or TDI line-scan camera.

### Sample Processing Methods

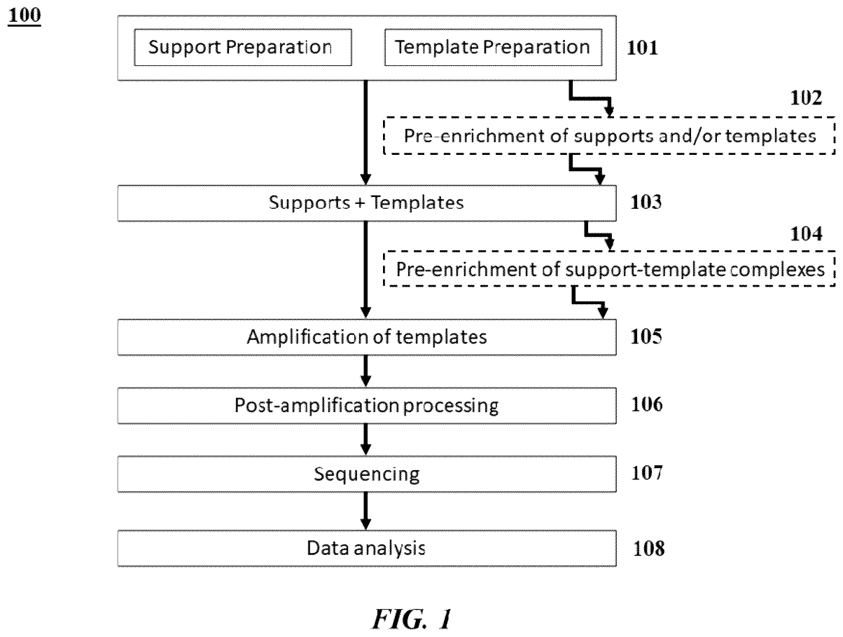

Described herein are devices, systems, methods, compositions, and kits for processing samples, such as to prepare a sample for sequencing, to sequence a sample, and/or to analyze sequencing data. FIG. 1 illustrates an example sequencing workflow 100, according to the devices, systems, methods, compositions, and kits of the present disclosure.

Supports and/or template nucleic acids may be prepared and/or provided (101) to be compatible with downstream sequencing operations (e.g., 107). A support (e.g., bead) may be used to help facilitate sequencing of a template nucleic acid on a substrate. The support may help immobilize a template nucleic acid to a substrate, such as when the template nucleic acid is coupled to the support, and the support is in turn immobilized to the substrate. The support may further function as a binding entity to retain molecules of a colony of the template nucleic acid (e.g., copies comprising identical or substantially identical sequences as the template nucleic acid) together for any downstream processing, such as for sequencing operations. This may be particularly useful in distinguishing a colony from other colonies (e.g., on other supports) and generating amplified sequencing signals for a template nucleic acid sequence.

A support that is prepared and/or provided may comprise an oligonucleotide comprising one or more functional nucleic acid sequences. For example, the support may comprise a capture sequence configured to capture or be coupled to a template nucleic acid (or processed template nucleic acid). For example, the support may comprise the capture sequence, a primer sequence, a barcode sequence, a sample index sequence, a unique molecular identifier (UMI), a flow cell adapter sequence, an adapter sequence, a binding sequence for any molecule (e.g., splint, primer, template nucleic acid, capture sequence, etc.), or any other functional sequence useful for a downstream operation, or any combination thereof. The oligonucleotide may be single-stranded, double-stranded, or partially double-stranded.

A support may comprise one or more capture entities, where a capture entity is configured for capture by a capturing entity. A capture entity may be coupled to an oligonucleotide coupled to the support. A capture entity may be coupled to the support. For example, the capturing entity may comprise streptavidin (SA) when the capture moiety comprises biotin. In another example, the capturing entity may comprise a complementary capture sequence when the capture entity comprises a capture sequence (e.g., a capture oligonucleotide that is complementary to the complementary capture sequence). In another example, the capturing entity may comprise an apparatus, system, or device configured to apply a magnetic field when the capture entity comprises a magnetic particle. In another example, the capturing entity may comprise an apparatus, system, or device configured to apply an electrical field when the capture entity comprises a charged particle. In some instances, the capturing entity may comprise one or more other mechanisms configured to capture the capture entity. A capture entity and capturing entity may bind, couple, hybridize, or otherwise associate with each other. The association may comprise formation of a covalent bond, non-covalent bond, and/or releasable bond (e.g., cleavable bond that is cleavable upon application of a stimulus). In some cases, the association may not form any bond. For example, the association may increase a physical proximity (or decrease a physical distance) between the capturing entity and capture entity. In some instances, a single capture entity may be capable of associating with a single capturing entity. Alternatively, a single capture entity may be capable of associating with multiple capturing entities. Alternatively or in addition, a single capturing entity may be capable of associating with multiple capture entities. The capture entity may be capable of linking to a nucleotide. Chemically modified bases comprising biotin, an azide, cyclooctyne, tetrazole, and a thiol, and many others are suitable as capture entities. The capture entity/capturing entity pair may be any combination. The pair may include, but is not limited to, biotin/streptavidin, azide/cyclooctyne, and thiol/maleimide. It will be appreciated that either of the pair may be used as either the capture entity or the capturing entity. In some instances, the capturing entity may comprise a secondary capture entity, for example, for subsequent capture by a secondary capturing entity. The secondary capture entity and secondary capturing entity may comprise any one or more of the capturing mechanisms described elsewhere herein (e.g., biotin and streptavidin, complementary capture sequences, etc.). In some instances, the secondary capture entity can comprise a magnetic particle (e.g., magnetic bead) and the secondary capturing entity can comprise a magnetic system (e.g., magnet, apparatus, system, or device configured to apply a magnetic field, etc.). In some instances, the secondary capture entity can comprise a charged particle (e.g., charged bead carrying an electrical charge) and the secondary capturing entity can comprise an electrical system (e.g., magnet, apparatus, system, or device configured to apply an electric field, etc.).

A support may comprise one or more cleaving moieties. The cleavable moiety may be part of or attached to an oligonucleotide coupled to the support. The cleavable moiety may be coupled to the support. A cleavable moiety may comprise any useful cleavable or excisable moiety that can be used to cleave an oligonucleotide (or portion thereof) from the support. For example, the cleavable moiety may comprise a uracil, a ribonucleotide, or other modified nucleotide that is excisable or cleavable using an enzyme (e.g., UDG, RNAse, endonuclease, exonuclease, etc.). The cleavable moiety may comprise an abasic site or an analog of an abasic site (e.g., dSpacer), a dideoxyribose. The cleavable moiety may comprise a spacer, e.g., C3 spacer, hexanediol, triethylene glycol spacer (e.g., Spacer 9), hexa-ethyleneglycol spacer (e.g., Spacer 18), or combinations or analogs thereof. The cleavable moiety may comprise a photocleavable moiety. The cleavable moiety may comprise a modified nucleotide, e.g., a methylated nucleotide. The modified nucleotide may be recognized specifically by an enzyme (e.g., a methylated nucleotide may be recognized by MspJI). The cleavable moiety may be cleaved enzymatically (e.g., using an enzyme such as UDG, RNAse, APE1, MspJI, etc.). Alternatively, or in addition to, the cleavable moiety may be cleavable using one or more stimuli, e.g., photo-stimulus, chemical stimulus, thermal stimulus, etc.

In some examples, a single support comprises copies of a single species of oligonucleotide, which are identical or substantially identical to each other. In some examples, a single support comprises copies of at least two species of oligonucleotides (e.g., comprising different sequences). For example, a single support may comprise a first subset of oligonucleotides configured to capture a first adapter sequence of a template nucleic acid and a second subset of oligonucleotides configured to capture a second adapter sequence of a template nucleic acid.

In some examples, a population of a single species of supports may be prepared and/or provided, where all supports within a species of supports is identical (e.g., has identical oligonucleotide composition (e.g., sequence), etc.). In some examples, a population of multiple species of supports may be prepared and/or provided. For example, a population of supports may be prepared to comprise a plurality of unique support species, where each unique support species comprises a primer sequence unique to the support species. When attaching template nucleic acids to supports, only a template nucleic acid comprising a given adapter sequence compatible with (e.g., at least partially complementary to) a given primer sequence may be capable of attaching to a given support of a support species comprising the given primer sequence. In another example, a population of supports may be prepared, such that each unique support species comprises a plurality of primer sequences (e.g., a pair of primer sequences) unique to the support species. In some embodiments, the systems and methods disclosed herein can include a population of supports that comprise two, three, four, five, six, seven, eight, nine, ten or more unique support species. Each unique support species can comprise a unique primer sequence that allows selective interactions between the respective support species with an intended binding partner (e.g., a complementary nucleic acid sequence within an adapter region of a template nucleic acid or an intermediary primer sequence which can subsequently bind to a complementary nucleic acid sequence within an adapter region of a sample nucleic acid). A population of multiple species of supports may be prepared by first preparing distinct populations of a single species of supports, all different, and mixing such distinct populations of single species of supports to result in the final population of multiple species of supports. A concentration of the different support species within the final mixture may be adjusted accordingly. Devices, systems, methods, compositions, and kits for preparing and using support species are described in further detail in International Pub. No. WO2020/167656 and International App. No. PCT/US2021/046951, each of which is entirely incorporated herein by reference for all purposes.

A template nucleic acid may include an insert sequence sourced from a biological sample. In some cases, the insert sequence may be derived from a larger nucleic acid in the biological sample (e.g., an endogenous nucleic acid), or reverse complement thereof, for example by fragmenting, transposing, and/or replicating from the larger nucleic acid. The template nucleic acid may be derived from any nucleic acid of the biological sample and result from any number of nucleic acid processing operations, such as but not limited to fragmentation, degradation or digestion, transposition, ligation, reverse transcription, extension, etc. A template nucleic acid that is prepared and/or provided may comprise one or more functional nucleic acid sequences. In some cases, the one or more functional nucleic acid sequences may be disposed at one end of the insert sequence. In some cases, the one or more functional nucleic acid sequences may be separated and disposed at both ends of an insert sequence, such as to sandwich the insert sequence. In some cases, a nucleic acid molecule comprising the insert sequence, or complement thereof, may be ligated to one or more adapter oligonucleotides that comprise such functional nucleic acid sequence(s). In some cases, a nucleic acid molecule comprising the insert sequence, or complement thereof, may be hybridized to a primer comprising such functional nucleic acid sequence(s) and extended to generate a template nucleic acid comprising such functional nucleic acid sequence(s). In some cases, a nucleic acid molecule comprising the insert sequence, or complement thereof, may be hybridized to a primer comprising one or more functional nucleic acid sequence(s) and extended to generate an intermediary molecule, and the intermediary molecule hybridized to a primer comprising additional functional nucleic acid sequence(s) and extended, and so on for any number of extension reactions, to generate a template nucleic acid comprising one or more functional nucleic acid sequence(s). For example, the template nucleic acid may comprise an adapter sequence configured to be captured by a capture sequence on an oligonucleotide coupled to a support. For example, the template nucleic acid may comprise a capture sequence, a primer sequence, a barcode sequence, a sample index sequence, a unique molecular identifier (UMI), a flow cell adapter sequence, the adapter sequence, a binding sequence for any molecule (e.g., splint, primer, template nucleic acid, capture sequence, etc.), or any other functional sequence useful for a downstream operation, or any combination thereof. The template nucleic acid may be single-stranded, double-stranded, or partially double-stranded.

A template nucleic acid may comprise one or more capture entities that are described elsewhere herein. In some cases, in the workflow, only the supports comprise capture entities and the template nucleic acids do not comprise capture entities. In other cases, in the workflow, only the template nucleic acids comprise capture entities and the supports do not comprise capture entities. In other cases, both the template nucleic acids and the supports comprise capture entities. In other cases, neither the supports nor the template nucleic acids comprises capture entities.

A template nucleic acid may comprise one or more cleaving moieties that are described elsewhere herein. In some cases, in the workflow, only the supports comprise cleavable moieties and the template nucleic acids do not comprise cleavable moieties. In other cases, in the workflow, only the template nucleic acids comprise cleavable moieties and the supports do not comprise cleavable moieties. In other cases, both the template nucleic acids and the supports comprise cleavable moieties. In other cases, neither the supports nor the template nucleic acids comprises cleavable moieties. A cleavable moiety may be strategically placed based on a desired downstream amplification workflow, for example.

In some examples, a library of insert sequences are processed to provide a population of template sequences with identical configurations, such as with identical sequences and/or locations of one or more functional sequences. For example, a population of template sequences may comprise a plurality of nucleic acid molecules each comprising an identical first adapter sequence ligated to a same end. In some examples, a library of insert sequences are processed to provide a population of template sequences with varying configurations, such as with varying sequences and/or locations of one or more functional sequences. For example, a population of template sequences may comprise a first subset of nucleic acid molecules each comprising an identical first adapter sequence at a first end, and a second subset of nucleic acid molecules each comprising an identical second adapter sequence at the second end, where the second adapter sequence is different form the first adapter sequence. In some instances, a population of template sequences with varying configurations (e.g., varying adapter sequences) may be used in conjunction with a population of multiple species of supports, such as to reduce polyclonality problems during downstream amplification. A population of multiple configurations of template nucleic acids may be prepared by first preparing distinct populations of a single configuration of template nucleic acids, all different, and mixing such distinct populations of single configurations of template nucleic acids to result in the final population of multiple configurations of template nucleic acids. A concentration of the different configurations of template nucleic acids within the final mixture may be adjusted accordingly.

Optionally, the supports and/or template nucleic acids may be pre-enriched (102). For example, a support comprising a distinct oligonucleotide sequence is isolated from a mixture comprising support(s) that do not have the distinct oligonucleotide sequence. Alternatively, a support population may be provided to comprise substantially uniform supports, where each support comprises an identical surface primer molecule immobilized thereto. For example, template nucleic acids comprising a distinct configuration (e.g., comprising a particular adapter sequence) is isolated from a mixture comprising template nucleic acids that do not have the distinct configuration. Alternatively, a template nucleic acid population may be provided to comprise substantially uniform configurations. In some cases, the capture entit(ies) on the supports and/or template nucleic acids are used for pre-enrichment.

Subsequent to preparation of the supports and template nucleic acids, the two may be attached (103). A template nucleic acid may be coupled to a support via any method(s) that results in a stable association between the template nucleic acid and the support. For example, the template nucleic acid may hybridize to an oligonucleotide on the support. In another example, the template nucleic acid may hybridize to one or more intermediary molecules, such as a splint, bridge, and/or primer molecule, which hybridizes to an oligonucleotide on the support. Alternatively or in addition, a template nucleic acid may be ligated to one or more nucleic acids on or coupled to the support. Alternatively or in addition, a template nucleic acid may be hybridized to an oligonucleotide on a support, which oligonucleotide comprises a primer sequence, and subsequent extension form the primer sequence is performed. Once attached, a plurality of support-template complexes may be generated.

Optionally, support-template complexes may be pre-enriched (104), wherein a support-template complex is isolated from a mixture comprising support(s) and/or template nucleic acid(s) that are not attached to each other. In some cases, the capture entit(ies) on the supports and/or template nucleic acids are used for pre-enrichment.

Subsequent to attachment of the template nucleic acid molecule to the support, the template nucleic acids may be subjected to amplification reactions (105) to generate a plurality of amplification products immobilized to the support. For example, such amplification reactions may comprise performing polymerase chain reaction (PCR) or any other amplification methods described herein, including but not limited to emulsion PCR (ePCR or emPCR), isothermal amplification (e.g., recombinase polymerase amplification (RPA)), bridge amplification, template walking, etc. In some cases, amplification reactions can occur while the support is immobilized to a substrate. In other cases, amplification reactions can occur off the substrate, such as in solution, or on a different surface or platform. In some cases, amplification reactions can occur in isolated reaction volumes, such as within multiple droplets in an emulsion during emulsion PCR (ePCR or emPCR), or in wells. Emulsion PCR methods are described in further detail in International Pub. No. WO2020/167656 and International App. No. PCT/US2021/046951, each of which is entirely incorporated by reference herein.

Subsequent to amplification, the supports (e.g., comprising the template nucleic acids) may be subjected to post-amplification processing (106). Often, subsequent to amplification, a resulting mixture may comprise a mix of positive supports (e.g., those comprising a template nucleic acid molecule) and negative supports (e.g., those not attached to template nucleic acid molecules). Enrichment procedure(s) may isolate positive supports from the mixtures. Example methods of enrichment of amplified supports are described in U.S. Pub. No. 2021/0277464 and International App. No. PCT/US2021/046951, each of which is entirely incorporated by reference herein. For example, an on-substrate enrichment procedure may immobilize only the positive supports onto the substrate surface to isolate the positive supports. In some instances, the positive supports may be immobilized to desired locations on the substrate surface (e.g., individually addressable locations), as distinguished from undesired locations (e.g., spacers between the individually addressable locations). In some instances, positive supports and/or negative supports may be processed to selectively remove unamplified surface primers (on the support(s)), such that a resulting positive support retains the template nucleic acid molecule, and a resulting negative support is stripped of the unamplified surface primers. Subsequently, the template nucleic acid(s) on the positive supports may be used to enrich for the positive supports, e.g., by capturing the template nucleic acids.

Subsequent to post-amplification processing, the template nucleic acids may be subject to sequencing (107). The template nucleic acid(s) may be sequenced while attached to the support. Alternatively, the template nucleic acid molecules may be free of the support when sequenced and/or analyzed. In some instances, the template nucleic acids may be sequenced while attached to the support which is immobilized to a substrate. Examples of substrate-based sample processing systems are described elsewhere herein. Any sequencing method described elsewhere herein may be used. In some cases, sequencing by synthesis (SBS) is performed.

In one example (Example A), an SBS method comprises flowing nucleotide reagents according to a flow order comprising a repeat of one 4-base flow (e.g., [A/T/G/C]), where each nucleotide is reversibly terminated (e.g., dideoxynucleotide), and where each base is labeled with a different dye (yielding different optical signals). With each flow, other sequencing reagents, e.g., sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of each base can be detected by interrogating the different dyes in 4 channels. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. In another example (Example B), an SBS method comprises flowing nucleotide reagents according to a flow order comprising a repeat of a flow cycle of 4 single base flows (e.g., [A T G C]), where each nucleotide is reversibly terminated, and where each base is labeled with a same dye (yielding same frequency optical signals). With each flow, other sequencing reagents, e.g., sequencing primer, polymerase, buffer, etc. are present to provide sufficient conditions for incorporation of the reversibly terminated, labeled nucleotide into a growing strand hybridized to a template nucleic acid. After each flow, an incorporation event or lack thereof of the particular base in that flow can be detected by interrogating the wavelength of the dye. After the incorporation events of a flow, in which at most one nucleotide is incorporated into each growing strand due to the terminated state, the termination can be reversed (e.g., cleaving a terminating moiety) to allow for subsequent stepwise incorporation events in subsequent flows. After each or one or more detection events, the labels may be removed (e.g., cleaved) to reduce signal noise for the next detection. In another example (Example C), an SBS method comprises flowing nucleotide reagents according to a flow order comprising a repeat of a flow cycle of 4 single base flows (e.g., [A T G C]), where each nucleotide is not terminated, and where each base is labeled with a same dye (yielding same frequency optical signals). With each flow, other sequencing reagents, e.g., sequencing primer, polymerase, buffer, etc. are present to provide sufficient conditions for incorporation of the labeled nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example D), an SBS method comprises flowing nucleotide reagents according to a flow order comprising a repeat of a flow cycle of 4 single base flows (e.g., [A T G C]), where each nucleotide is not terminated, and where only a fraction of the bases in each flow (e.g., less than 90%, 80%, 70%, 60%, 50%, 40%, 30%, 20%, 10%, 9%, 8%, 7%, 6%, 5%, 4%, 3%, 2%, 1%, etc.) is labeled with a same dye (yielding same frequency optical signals). With each flow, other sequencing reagents, e.g., sequencing primer, polymerase, buffer, etc. are present to provide sufficient conditions for incorporation of the nucleotide into a growing strand hybridized to a template nucleic acid. After each flow, an incorporation event or lack thereof of the particular base in that flow can be detected by interrogating the wavelength of the dye. Because the nucleotides are not terminated, if the growing strand is extending through a homopolymer region (e.g., polyT region, etc.) of the template nucleic acid, multiple nucleotides may be incorporated during one flow. After each or one or more detection events, the labels may be removed (e.g., dyes are cleaved) to reduce signal noise for the next detection. In another example (Example E), an SBS method comprises flowing nucleotide reagents according to a flow order comprising a repeat of a flow cycle of 8 single base flows, with each of the 4 canonical base types flowed twice consecutively within the flow cycle, (e.g., [A A T T G G C C]), where each nucleotide is not terminated, and where only a fraction of the bases in every other flow in the flow cycle (e.g., less than 90%, 80%, 70%, 60%, 50%, 40%, 30%, 20%, 10%, 9%, 8%, 7%, 6%, 5%, 4%, 3%, 2%, 1%, etc.) is labeled with a same dye (yielding same frequency optical signals) and the nucleotides in the alternating other flow is unlabeled. With each flow, other sequencing reagents, e.g., sequencing primer, polymerase, buffer, etc. are present to provide sufficient conditions for incorporation of the nucleotide into a growing strand hybridized to a template nucleic acid. After one or both of the flows for each canonical base type, an incorporation event or lack thereof of the particular base in that flow can be detected by interrogating the wavelength of the dye. Because the nucleotides are not terminated, if the growing strand is extending through a homopolymer region (e.g., polyT region) of the template nucleic acid, multiple nucleotides may be incorporated during one flow. A first flow of a canonical base type (e.g., A) followed by a second flow of the same canonical base type (e.g., A) may help facilitate completion of incorporation reactions across each growing strand such as to reduce phasing problems. After each or one or more detection events, the labels may be removed (e.g., dyes are cleaved) to reduce signal noise for the next detection.

Labeled nucleotides may comprise a dye, fluorophore, or quantum dot. Non-limiting examples of dyes include SYBR green, SYBR blue, DAPI, propidium iodine, Hoechst, SYBR gold, ethidium bromide, acridine, proflavine, acridine orange, acriflavine, fluorocoumarin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), VIC, 5- (or 6-) iodoacetamidofluorescein, 5-{[2(and 3)-5-(Acetylmercapto)-succinyl]amino}fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, Atto 390, 425, 465, 488, 495, 532, 565, 594, 633, 647, 647N, 665, 680 and 700 dyes, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores, Black Hole Quencher Dyes (Biosearch Technologies) such as BH1-0, BHQ-1, BHQ-3, BHQ-10); QSY Dye fluorescent quenchers (from Molecular Probes/Invitrogen) such QSY7, QSY9, QSY21, QSY35, and other quenchers such as Dabcyl and Dabsyl; Cy5Q and Cy7Q and Dark Cyanine dyes (GE Healthcare); Dy-Quenchers (Dyomics), such as DYQ-660 and DYQ-661; and ATTO fluorescent quenchers (ATTO-TEC GmbH), such as ATTO 540Q, 580Q, 612Q. In some cases, the label may be one with linkers. For instance, a label may have a disulfide linker attached to the label. Non-limiting examples of such labels include Cy5-azide, Cy-2-azide, Cy-3-azide, Cy-3.5-azide, Cy5.5-azide and Cy-7-azide. In some cases, a linker may be a cleavable linker. In some cases, the label may be a type that does not self-quench or exhibit proximity quenching. Non-limiting examples of a label type that does not self-quench or exhibit proximity quenching include Bimane derivatives such as Monobromobimane. Alternatively, the label may be a type that self-quenches or exhibits proximity quenching. Non-limiting examples of such labels include Cy5-azide, Cy-2-azide, Cy-3-azide, Cy-3.5-azide, Cy5.5-azide and Cy-7-azide. In some instances, a blocking group of a reversible terminator may comprise the dye.

It will be appreciated that the combinations of termination states on the nucleotides, label types (e.g., types of dye or other detectable moiety), fraction of labeled nucleotides within a flow, type of nucleotide bases in each flow, type of nucleotide bases in each flow cycle, and/or the order of flows in a flow cycle and/or flow order, other than enumerated in Examples A-E, can be varied for different SBS methods.

Subsequent to sequencing, the sequencing signals collected and/or generated may be subjected to data analysis (108). The sequencing signals may be processed to generate base calls and/or sequencing reads. In some cases, the sequencing reads may be processed to generate diagnostics data to the biological sample, or the subject from which the biological sample was derived from.

While the sequencing workflow 100 with respect to FIG. 1 has been described with respect to the use of supports to bind template molecules, it will be appreciated that the different supports may be effectively replaced by using spatially distinct locations on one or more surfaces, which do not necessarily have to be the surfaces of individual supports (e.g., beads). For example, a first spatially distinct location on a surface may be capable of directly immobilizing a first colony of a first template nucleic acid and a second spatially distinct location on the same surface (or a different surface) may be capable of directly immobilizing a second colony of a second template nucleic acid to distinguish from the first colony. In some cases, the surface comprising the spatially distinct locations may be a surface of the substrate on which the sample is sequenced, thus streamlining the amplification-sequencing workflow.

It will be appreciated that in some instances, the different operations described in the sequencing workflow 100 may be performed in a different order. It will be appreciated that in some instances, one or more operations described in the sequencing workflow 100 may be omitted or replaced with other comparable operation(s). It will be appreciated that in some instances, one or more additional operations described in the sequencing workflow 100 may be performed.

The different operations described with respect to sequencing workflow 100 may be performed with the help of open substrate systems described herein.

### Open Substrate Systems

Described herein are devices, systems, and methods that use open substrates or open flow cell geometries to process a sample. The term “open substrate,” as used herein, generally refers to a substrate in which any point on an active surface of the substrate is physically accessible from a direction normal to the substrate. The devices, systems and methods may be used to facilitate any application or process involving a reaction or interaction between two objects, such as between an analyte and a reagent or between two reagents. For example, the reaction or interaction may be chemical (e.g., polymerase reaction) or physical (e.g., displacement). The devices, systems, and methods described herein may benefit from higher efficiency, such as from faster reagent delivery and lower volumes of reagents required per surface area. The devices, systems, and methods described herein may avoid contamination problems common to microfluidic channel flow cells that are fed from multiport valves which can be a source of carryover from one reagent to the next. The devices, systems, and methods may benefit from shorter completion time, use of fewer resources (e.g., various reagents), and/or reduced system costs. The open substrates or flow cell geometries may be used to process any analyte from any sample, such as but not limited to, nucleic acid molecules, protein molecules, antibodies, antigens, cells, and/or organisms, as described herein. The open substrates or flow cell geometries may be used for any application or process, such as, but not limited to, sequencing by synthesis, sequencing by ligation, amplification, proteomics, single cell processing, barcoding, and sample preparation, as described herein.

A sample processing system may comprise a substrate, and devices and systems that perform one or more operations with or on the substrate. The sample processing system may permit highly efficient dispensing of reagents onto the substrate. The sample processing may permit highly efficient imaging of one or more analytes, or signals corresponding thereto, on the substrate. The sample processing system may comprise an imaging system comprising a detector. Substrates and detectors that can be used in the sample processing system are described in further detail in International Pub. No. WO2019/099886, U.S. Pub. No. 2021/0354126, and U.S. Pub. No. 2021/0277464, each of which is entirely incorporated herein by reference for all purposes.

### Substrates

The substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SiₓO_{y}, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating. The substrate may comprise multiple layers of the same or different type of material. The substrate may be fully or partially opaque to visible light. The substrate may be fully or partially transparent to visible light. A surface of the substrate may be modified to comprise active chemical groups, such as amines, esters, hydroxyls, epoxides, and the like, or a combination thereof. A surface of the substrate may be modified to comprise any of the binders or linkers described herein. In some instances, such binders, linkers, active chemical groups, and the like may be added as an additional layer or coating to the substrate.

The substrate may have the general form of a cylinder, a cylindrical shell or disk, a rectangular prism, or any other geometric form. The substrate may have a thickness (e.g., a minimum dimension) of at least 100 micrometers (μm), at least 200 μm, at least 500 μm, at least 1 mm, at least 2 millimeters (mm), at least 5 mm, at least 10 mm, or more. The substrate may have a first lateral dimension (such as a width for a substrate having the general form of a rectangular prism or a radius or diameter for a substrate having the general form of a cylinder) and/or a second lateral dimension (such as a length for a substrate having the general form of a rectangular prism) of at least 1 mm, at least 2 mm, at least 5 mm, at least 10 mm, at least 20 mm, at least 50 mm, at least 100 mm, at least 200 mm, at least 500 mm, at least 1,000 mm, or more.

One or more surfaces of the substrate may be exposed to a surrounding open environment, and accessible from such surrounding open environment. For example, the array may be exposed and accessible from such surrounding open environment. In some cases, as described elsewhere herein, the surrounding open environment may be controlled and/or confined in a larger controlled environment.

The substrate may comprise a plurality of individually addressable locations. The individually addressable locations may comprise locations that are physically accessible for manipulation. The manipulation may comprise, for example, placement, extraction, reagent dispensing, seeding, heating, cooling, or agitation. The manipulation may be accomplished through, for example, localized microfluidic, pipet, optical, laser, acoustic, magnetic, and/or electromagnetic interactions with the analyte or its surroundings. The individually addressable locations may comprise locations that are digitally accessible. For example, each individually addressable location may be located, identified, and/or accessed electronically or digitally for indexing, mapping, sensing, associating with a device (e.g., detector, processor, dispenser, etc.), or otherwise processing.

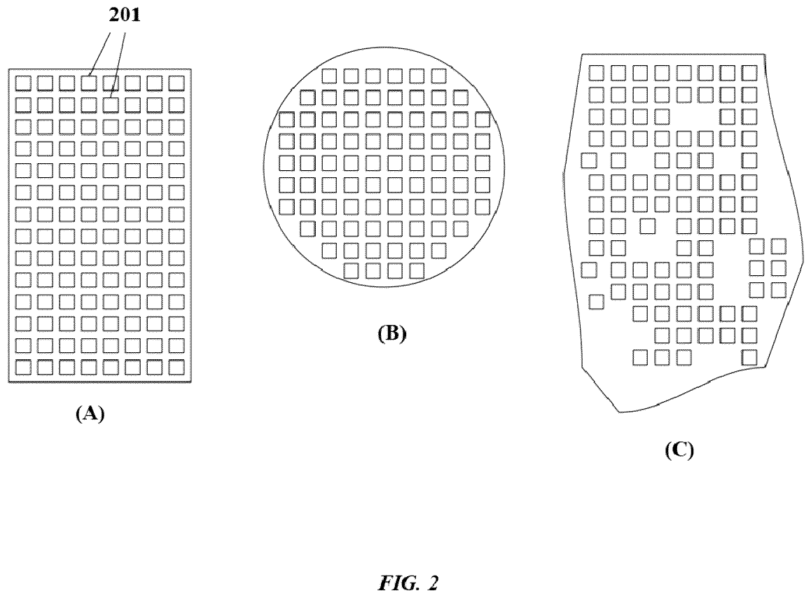

The plurality of individually addressable locations may be arranged as an array, randomly, or according to any pattern, on the substrate. FIG. 2 illustrates different substrates (from a top view) comprising different arrangements of individually addressable locations 201, with panel A showing a substantially rectangular substrate with regular linear arrays, panel B showing a substantially circular substrate with regular linear arrays, and panel C showing an arbitrarily shaped substrate with irregular arrays. The substrate may have any number of individually addressable locations, for example, at least 1, at least 2, at least 5, at least 10, at least 20, at least 50, at least 100, at least 200, at least 500, at least 1,000, at least 2,000, at least 5,000, at least 10,000, at least 20,000, at least 50,000, at least 100,000, at least 200,000, at least 500,000, at least 1,000,000, at least 2,000,000, at least 5,000,000, at least 10,000,000, at least 20,000,000, at least 50,000,000, at least 100,000,000, at least 200,000,000, at least 500,000,000, at least 1,000,000,000, at least 2,000,000,000, at least 5,000,000,000, at least 10,000,000,000, at least 20,000,000,000, at least 50,000,000,000, at least 100,000,000,000 or more individually addressable locations. The substrate may have a number of individually addressable locations that is within a range defined by any two of the preceding values.

Each individually addressable location may have the general shape or form of a circle, pit, bump, rectangle, or any other shape or form (e.g., polygonal, non-polygonal). A plurality of individually addressable locations can have uniform shape or form, or different shapes or forms. An individually addressable location may have any size. In some cases, an individually addressable location may have an area of about 0.1 square micron (μm²), about 0.2 μm², about 0.25 μm², about 0.3 μm², about 0.4 μm², about 0.5 μm², about 0.6 μm², about 0.7 μm², about 0.8 μm², about 0.9 μm², about 1 μm², about 1.1 μm², about 1.2 μm², about 1.25 μm², about 1.3 μm², about 1.4 μm², about 1.5 μm², about 1.6 μm², about 1.7 μm², about 1.75 μm², about 1.8 μm², about 1.9 μm², about 2 μm², about 2.25 μm², about 2.5 μm², about 2.75 μm², about 3 μm², about 3.25 μm², about 3.5 μm², about 3.75 μm², about 4 μm², about 4.25 μm², about 4.5 μm², about 4.75 μm², about 5 μm², about 5.5 μm², about 6 μm², or more. An individually addressable location may have an area that is within a range defined by any two of the preceding values. An individually addressable location may have an area that is less than about 0.1 μm² or greater than about 6 μm².

The individually addressable locations may be distributed on a substrate with a pitch determined by the distance between the center of a first location and the center of the closest or neighboring individually addressable location. Locations may be spaced with a pitch of about 0.1 micron (μm), about 0.2 μm, about 0.25 μm, about 0.3 μm, about 0.4 μm, about 0.5 μm, about 0.6 μm, about 0.7 μm, about 0.8 μm, about 0.9 μm, about 1 μm, about 1.1 μm, about 1.2 μm, about 1.25 μm, about 1.3 μm, about 1.4 μm, about 1.5 μm, about 1.6 μm, about 1.7 μm, about 1.75 μm, about 1.8 μm, about 1.9 μm, about 2 μm, about 2.25 μm, about 2.5 μm, about 2.75 μm, about 3 μm, about 3.25 μm, about 3.5 μm, about 3.75 μm, about 4 μm, about 4.25 μm, about 4.5 μm, about 4.75 μm, about 5 μm, about 5.5 μm, about 6 μm, about 6.5 μm, about 7 μm, about 7.5 μm, about 8 μm, about 8.5 μm, about 9 μm, about 9.5 μm, or about 10 μm. In some cases, the locations may be positioned with a pitch that is within a range defined by any two of the preceding values. The locations may be positioned with a pitch of less than about 0.1 μm or greater than about 10 μm. In some cases, the pitch between two individually addressable locations may be determined as a function of a size of a loading object (e.g., bead). For example, where the loading object is a bead having a maximum diameter, the pitch may be at least about the maximum diameter of the loading object.

Each of the plurality of individually addressable locations, or each of a subset of such locations, may be capable of immobilizing thereto an analyte (e.g., a nucleic acid molecule, a protein molecule, a carbohydrate molecule, etc.) or a reagent (e.g., a nucleic acid molecule, a probe molecule, a barcode molecule, an antibody molecule, a primer molecule, a bead, etc.). In some cases, an analyte or reagent may be immobilized to an individually addressable location via a support, such as a bead. In an example, a bead is immobilized to the individually addressable location, and the analyte or reagent is immobilized to the bead. In some cases, an individually addressable location may immobilize thereto a plurality of analytes or a plurality of reagents, such as via the support. The substrate may immobilize a plurality of analytes or reagents across multiple individually addressable locations. The plurality of analytes or reagents may be of the same type of analyte or reagent (e.g., a nucleic acid molecule) or may be a combination of different types of analytes or reagents (e.g., nucleic acid molecules, protein molecules, etc.). In an example, a first bead comprising a first colony of nucleic acid molecules each comprising a first template sequence is immobilized to a first individually addressable location, and a second bead comprising a second colony of nucleic acid molecules each comprising a second template sequence is immobilized to a second individually addressable location.

A substrate may comprise more than one type of individually addressable location arranged as an array, randomly, or according to any pattern, on the substrate. In some cases, different types of individually addressable locations may have different chemical, physical, and/or biological properties (e.g., hydrophobicity, charge, color, topography, size, dimensions, geometry, etc.). For example, a first type of individually addressable location may bind a first type of biological analyte but not a second type of biological analyte, and a second type of individually addressable location may bind the second type of biological analyte but not the first type of biological analyte.

In some cases, an individually addressable location may comprise a distinct surface chemistry. The distinct surface chemistry may distinguish between different addressable locations. The distinct surface chemistry may distinguish an individually addressable location from a surrounding location on the substrate. For example, a first location type may comprise a first surface chemistry, and a second location type may lack the first surface chemistry. In another example, the first location type may comprise the first surface chemistry and the second location type may comprise a second, different surface chemistry. A first location type may have a first affinity towards an object (e.g., a bead comprising nucleic acid molecules, e.g., amplicons, immobilized thereto) and a second location type may have a second, different affinity towards the same object due to different surface chemistries. In other examples, a first location type comprising a first surface chemistry may have an affinity towards a first sample type (e.g., a bead comprising nucleic acid molecules, e.g., amplicons, immobilized thereto) and exclude a second sample type (e.g., a bead lacking nucleic acid molecules, e.g., amplicons, immobilized thereto). The first location type and the second location type may or may not be disposed on the surface in alternating fashion. For example, a first location type or region type may comprise a positively charged surface chemistry and a second location type or region type may comprise a negatively charged surface chemistry. In another example, a first location type or region type may comprise a hydrophobic surface chemistry and a second location type or region type may comprise a hydrophilic surface chemistry. In another example, a first location type comprises a binder, as described elsewhere herein, and a second location type does not comprise the binder or comprises a different binder. In some cases, a surface chemistry may comprise an amine. In some cases, a surface chemistry may comprise a silane (e.g., tetramethylsilane). In some cases, the surface chemistry may comprise hexamethyldisilazane (HMDS). In some cases, the surface chemistry may comprise (3-aminopropyl)triethoxysilane (APTMS). In some cases, the surface chemistry may comprise a surface primer molecule or any oligonucleotide molecule that has any degree of affinity towards another molecule. In one example, the substrate comprises a plurality of individually addressable locations, each defined by APTMS, which are positively charged and has affinity towards an amplified bead (e.g., a bead comprising nucleic acid molecules, e.g., amplicons, immobilized thereto) which exhibits a negative charge. The locations surrounding the plurality of individually addressable locations may comprise HMDS which repels amplified beads.

In some cases, the individually addressable locations may be indexed, e.g., spatially. Data corresponding to an indexed location, collected over multiple periods of time, may be linked to the same indexed location. In some cases, sequencing signal data collected from an indexed location, during iterations of sequencing-by-synthesis flows, are linked to the indexed location to generate a sequencing read for an analyte immobilized at the indexed location. In some embodiments, the individually addressable locations are indexed by demarcating part of the surface, such as by etching or notching the surface, using a dye or ink, depositing a topographical mark, depositing a sample (e.g., a control nucleic acid sample), depositing a reference object (e.g., e.g., a reference bead that always emits a detectable signal during detection), and the like, and the individually addressable locations may be indexed with reference to such demarcations. As will be appreciated, a combination of positive demarcations and negative demarcations (lack thereof) may be used to index the individually addressable locations. In some embodiments, each of the individually addressable locations is indexed. In some embodiments, a subset of the individually addressable locations is indexed. In some embodiments, the individually addressable locations are not indexed, and a different region of the substrate is indexed.

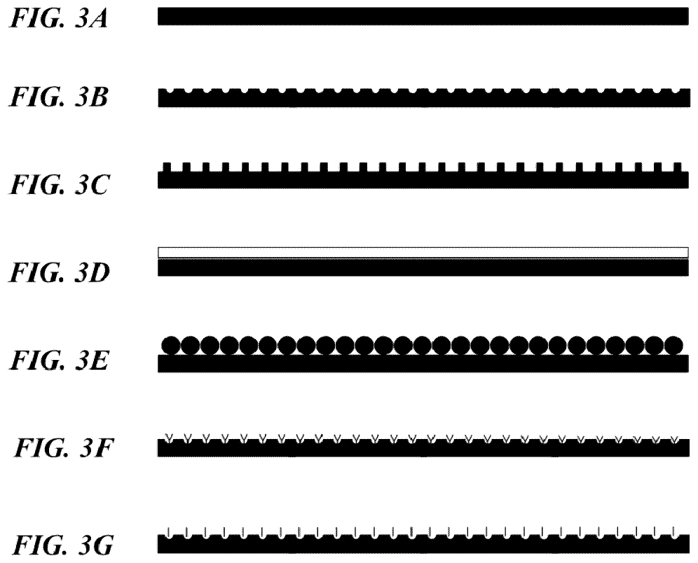

The substrate may comprise a planar or substantially planar surface. Substantially planar may refer to planarity at a micrometer level (e.g., a range of unevenness on the planar surface does not exceed the micrometer scale) or nanometer level (e.g., a range of unevenness on the planar surface does not exceed the nanometer scale). Alternatively, substantially planar may refer to planarity at less than a nanometer level or greater than a micrometer level (e.g., millimeter level). Alternatively or in addition, a surface of the substrate may be textured or patterned. For example, the substrate may comprise grooves, troughs, hills, and/or pillars. The substrate may define one or more cavities (e.g., micro-scale cavities or nano-scale cavities). The substrate may define one or more channels. The substrate may have regular textures and/or patterns across the surface of the substrate. For example, the substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. In some instances, a texture of the substrate may comprise structures having a maximum dimension of at most about 100%, 90%, 80%, 70%, 60%, 50%, 40%, 30%, 20%, 10%, 9%, 8%, 7%, 6%, 5%, 4%, 3%, 2%, 1%, 0.1%, 0.01%, 0.001%, 0.0001%, 0.00001% of the total thickness of the substrate or a layer of the substrate. In some instances, the textures and/or patterns of the substrate may define at least part of an individually addressable location on the substrate. A textured and/or patterned substrate may be substantially planar. FIGS. 3A-3G illustrate different examples of cross-sectional surface profiles of a substrate. FIG. 3A illustrates a cross-sectional surface profile of a substrate having a completely planar surface. FIG. 3B illustrates a cross-sectional surface profile of a substrate having semi-spherical troughs or grooves. FIG. 3C illustrates a cross-sectional surface profile of a substrate having pillars, or alternatively or in conjunction, wells. FIG. 3D illustrates a cross-sectional surface profile of a substrate having a coating. FIG. 3E illustrates a cross-sectional surface profile of a substrate having spherical particles. FIG. 3F illustrates a cross-sectional surface profile of FIG. 3B, with a first type of binders seeded or associated with the respective grooves. FIG. 3G illustrates a cross-sectional surface profile of FIG. 3B, with a second type of binders seeded or associated with the respective grooves.

A binder may be configured to immobilize an analyte or reagent to an individually addressable location. In some cases, a surface chemistry of an individually addressable location may comprise one or more binders. In some cases, a plurality of individually addressable locations may be coated with binders. In some cases, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, or at least 99% of the total number of individually addressable locations, or of the surface area of the substrate, are coated with binders. The binders may be integral to the array. The binders may be added to the array. For instance, the binders may be added to the array as one or more coating layers on the array. The substrate may comprise an order of magnitude of at least about 10, 100, 10³, 10⁴, 10⁵, 10⁶, 10⁷, 10⁸, 10⁹, 10¹⁰, 10¹¹, or more binders. Alternatively or in addition, the substrate may comprise an order of magnitude of at most about 10¹¹, 10¹⁰, 10⁹, 10⁸, 10⁷, 10⁶, 10⁵, 10⁴, 10³, 100, 10 or fewer binders.

The binders may immobilize analytes or reagents through non-specific interactions, such as one or more of hydrophilic interactions, hydrophobic interactions, electrostatic interactions, physical interactions (for instance, adhesion to pillars or settling within wells), and the like. Alternatively or in addition, the binders may immobilize analytes or reagents through specific interactions. For instance, where the analyte or reagent is a nucleic acid molecule, the binders may comprise oligonucleotide adaptors configured to bind to the nucleic acid molecule. In other examples, the binders may comprise one or more of antibodies, oligonucleotides, nucleic acid molecules, aptamers, affinity binding proteins, lipids, carbohydrates, and the like. The binders may immobilize analytes or reagents through any possible combination of interactions. For instance, the binders may immobilize nucleic acid molecules through a combination of physical and chemical interactions, through a combination of protein and nucleic acid interactions, etc. In some instances, a single binder may bind a single analyte (e.g., nucleic acid molecule) or single reagent. In some instances, a single binder may bind a plurality of analytes (e.g., plurality of nucleic acid molecules) or a plurality of reagents. In some instances, a plurality of binders may bind a single analyte or a single reagent. Though examples herein describe interactions of binders with nucleic acid molecules, the binders may immobilize other molecules (such as proteins), other particles, cells, viruses, other organisms, or the like. Though examples herein describe interactions of binders with samples or analytes, the binders may similarly immobilize reagents. In some instances, the substrate may comprise a plurality of types of binders, for example to bind different types of analytes or reagents. For example, a first type of binders (e.g., oligonucleotides) are configured to bind a first type of analyte (e.g., nucleic acid molecules) or reagent, and a second type of binders (e.g., antibodies) are configured to bind a second type of analyte (e.g., proteins) or reagent. In another example, a first type of binders (e.g., first type of oligonucleotide molecules) are configured to bind a first type of nucleic acid molecules and a second type of binders (e.g., second type of oligonucleotide molecules) are configured to bind a second type of nucleic acid molecules. For example, the substrate may be configured to bind different types of analytes or reagents in certain fractions or specific locations on the substrate by having the different types of binders in the certain fractions or specific locations on the substrate.

The substrate may be rotatable about an axis. The axis of rotation may or may not be an axis through the center of the substrate. In some instances, the systems, devices, and apparatus described herein may further comprise an automated or manual rotational unit configured to rotate the substrate. The rotational unit may comprise a motor and/or a rotor to rotate the substrate. For instance, the substrate may be affixed to a chuck (such as a vacuum chuck). The substrate may be rotated at a rotational speed of at least 1 revolution per minute (rpm), at least 2 rpm, at least 5 rpm, at least 10 rpm, at least 20 rpm, at least 50 rpm, at least 100 rpm, at least 200 rpm, at least 500 rpm, at least 1,000 rpm, at least 2,000 rpm, at least 5,000 rpm, at least 10,000 rpm, or greater. Alternatively or in addition, the substrate may be rotated at a rotational speed of at most about 10,000 rpm, 5,000 rpm, 2,000 rpm, 1,000 rpm, 500 rpm, 200 rpm, 100 rpm, 50 rpm, 20 rpm, 10 rpm, 5 rpm, 2 rpm, 1 rpm, or less. The substrate may be configured to rotate with a rotational velocity that is within a range defined by any two of the preceding values. The substrate may be configured to rotate with different rotational velocities during different operations described herein. The substrate may be configured to rotate with a rotational velocity that varies according to a time-dependent function, such as a ramp, sinusoid, pulse, or other function or combination of functions. The time-varying function may be periodic or aperiodic.

Analytes or reagents may be immobilized to the substrate during rotation. Analytes or reagents may be dispensed onto the substrate prior to or during rotation of the substrate. When the substrate is rotated at a relatively high rotational velocity, high speed coating across the substrate may be achieved via tangential inertia directing unconstrained spinning reagents in a partially radial direction (that is, away from the axis of rotation) during rotation, a phenomenon commonly referred to as centrifugal force. In some cases, the substrate may be rotated at relatively low velocities such that reagents dispensed to a certain location do not move to another location, or moves minimally, because of the rotation, to permit controlled dispensing of reagents to desired locations. For controlled dispensing, the substrate may be rotating with a rotational frequency of no more than 60 rpm, no more than 50 rpm, no more than 40 rpm, no more than 30 rpm, no more than 25 rpm, no more than 20 rpm, no more than 15 rpm, no more than 14 rpm, no more than 13 rpm, no more than 12 rpm, no more than 11 rpm, no more than 10 rpm, no more than 9 rpm, no more than 8 rpm, no more than 7 rpm, no more than 6 rpm, no more than 5 rpm, no more than 4 rpm, no more than 3 rpm, no more than 2 rpm, or no more than 1 rpm. In some cases the rotational frequency may be within a range defined by any two of the preceding values. In some cases the substrate may be rotating with a rotational frequency of about 5 rpm during controlled dispensing. A speed of substrate rotation may be adjusted according to the appropriate operation (e.g., high speed for spin-coating, high speed for washing the substrate, low speed for sample loading, low speed for detection, etc.).

In some cases, the substrate may be movable in any vector or direction. For example, such motion may be non-linear (e.g., in rotation about an axis), linear, or a hybrid of linear and non-linear motion. In some instances, the systems, devices, and apparatus described herein may further comprise a motion unit configured to move the substrate. The motion unit may comprise any mechanical component, such as a motor, rotor, actuator, linear stage, drum, roller, pulleys, etc., to move the substrate. Analytes or reagents may be immobilized to the substrate during any such motion. Analytes or reagents may be dispensed onto the substrate prior to, during, or subsequent to motion of the substrate.

Loading Reagents onto an Open Substrate

The surface of the substrate may be in fluid communication with at least one fluid nozzle (of a fluid channel). The surface may be in fluid communication with the fluid nozzle via a non-solid gap, e.g., an air gap. In some cases, the surface may additionally be in fluid communication with at least one fluid outlet. The surface may be in fluid communication with the fluid outlet via an air gap. The nozzle may be configured to direct a solution to the array. The outlet may be configured to receive a solution from the substrate surface. The solution may be directed to the surface using one or more dispensing nozzles. For example, the solution may be directed to the array using at least 1, 2, 3, 4, 5, 6, 7, 8, 9, 10, 11, 12, 13, 14, 15, 16, 17, 18, 19, 20 or more dispensing nozzles. The solution may be directed to the array using a number of nozzles that is within a range defined by any two of the preceding values. In some cases, different reagents (e.g., nucleotide solutions of different types, different probes, washing solutions, etc.) may be dispensed via different nozzles, such as to prevent contamination. Each nozzle may be connected to a dedicated fluidic line or fluidic valve, which may further prevent contamination. A type of reagent may be dispensed via one or more nozzles. The one or more nozzles may be directed at or in proximity to a center of the substrate. Alternatively, the one or more nozzles may be directed at or in proximity to a location on the substrate other than the center of the substrate. Alternatively or in combination, one or more nozzles may be directed closer to the center of the substrate than one or more of the other nozzles. For instance, one or more nozzles used for dispensing washing reagents may be directed closer to the center of the substrate than one or more nozzles used for dispensing active reagents. The one or more nozzles may be arranged at different radii from the center of the substrate. Two or more nozzles may be operated in combination to deliver fluids to the substrate more efficiently. One or more nozzles may be configured to deliver fluids to the substrate as a jet, spray (or other dispersed fluid), and/or droplets. One or more nozzles may be operated to nebulize fluids prior to delivery to the substrate. For example, the fluids may be delivered as aerosol particles.

In some cases, the solution may be dispensed on the substrate while the substrate is stationary; the substrate may then be subjected to rotation (or other motion) following the dispensing of the solution. Alternatively, the substrate may be subjected to rotation (or other motion) prior to the dispensing of the solution; the solution may then be dispensed on the substrate while the substrate is rotating (or otherwise moving). In some cases, rotation of the substrate may yield a centrifugal force (or inertial force directed away from the axis) on the solution, causing the solution to flow radially outward over the array. In this manner, rotation of the substrate may direct the solution across the array. Continued rotation of the substrate over a period of time may dispense a fluid film of a nearly constant thickness across the array.

One or more conditions such as the rotational velocity of the substrate, the acceleration of the substrate (e.g., the rate of change of velocity), viscosity of the solution, angle of dispensing (e.g., contact angle of a stream of reagents) of the solution, radial coordinates of dispensing of the solution (e.g., on center, off center, etc.), temperature of the substrate, temperature of the solution, and other factors may be adjusted and/or otherwise optimized to attain a desired wetting on the substrate and/or a film thickness on the substrate, such as to facilitate uniform coating of the substrate. For instance, one or more conditions may be applied to attain a film thickness of at least 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm), or more. Alternatively or in addition, one or more conditions may be applied to attain a film thickness of at most 10 nanometers (nm), 20 nm, 50 nm, 100 nm, 200 nm, 500 nm, 1 micrometer (μm), 2 μm, 5 μm, 10 μm, 20 μm, 50 μm, 100 μm, 200 μm, 500 μm, 1 millimeter (mm) or less. One or more conditions may be applied to attain a film thickness that is within a range defined by any two of the preceding values. The thickness of the film may be measured or monitored by a variety of techniques, such as thin film spectroscopy with a thin film spectrometer, such as a fiber spectrometer. In some cases, a surfactant may be added to the solution, or a surfactant may be added to the surface to facilitate uniform coating or to facilitate sample loading efficiency. Alternatively or in conjunction, the thickness of the solution may be adjusted using mechanical, electric, physical, or other mechanisms. For example, the solution may be dispensed onto a substrate and subsequently leveled using, e.g., a physical scraper such as a squeegee, to obtain a desired thickness of uniformity across the substrate.

Reagents may be dispensed to the substrate to multiple locations, and/or multiple reagents may be dispensed to the substrate to a single location, via different mechanisms. Reagent dispensing mechanisms disclosed herein may be applicable to sample dispensing. For example, a reagent may comprise the sample. The term “loading onto a substrate,” as used in reference to a reagent or a sample herein, may refer to dispensing of the reagent or the sample to a surface of the substrate in accordance with any reagent dispensing mechanism described herein.

In some cases, dispensing may be achieved via relative motion of the substrate and the dispenser (e.g., nozzle). For example, a reagent may be dispensed to the substrate at a first location, and thereafter travel to a second location different from the first location due to forces (e.g., centrifugal forces, centripetal forces, inertial forces, etc.) caused by motion of the substrate (e.g., rotational motion of the substrate, linear motion of the substrate, combination thereof, etc.). In another example, a reagent may be dispensed to a reference location, and the substrate may be moved relative to the reference location such that the reagent is dispensed to multiple locations of the substrate. In another example, a dispenser may be moved relative to the substrate to dispense the reagent at different locations, for example moved prior to, during, or subsequent to dispensing. In an example, a reagent is ‘painted’ onto the substrate by moving the dispenser and/or the substrate relative to each other, along a desired path on the substrate. The open substrate geometry may allow for flexible and controlled dispensing of a reagent to a desired location on the substrate. In some cases, dispensing may be achieved without relative motion between the substrate and the dispenser. For example, multiple dispensers may be used to dispense reagents to different locations, and/or multiple reagents to a single location, or a combination thereof (e.g., multiple reagents to multiple locations).

In another example, an external force (e.g., involving a pressure differential, involving physical force, involving a magnetic force, involving an electrical force, etc.), such as wind, a field-generating device, or a physical device, may be applied to one or more surfaces of the substrate to direct reagents to different locations across the substrate. In another example, the method for dispensing reagents may comprise vibration. In such an example, reagents may be distributed or dispensed onto a single region or multiple regions of the substrate (or a surface of the substrate). The substrate (or a surface thereof) may then be subjected to vibration, which may spread the reagent to different locations across the substrate (or the surface). Alternatively or in conjunction, the method may comprise using mechanical, electric, physical, or other mechanisms to dispense reagents to the substrate. For example, the solution may be dispensed onto a substrate and a physical scraper (e.g., a squeegee) may be used to spread the dispensed material or spread the reagents to different locations and/or to obtain a desired thickness or uniformity across the substrate. Beneficially, such flexible dispensing may be achieved without contamination of the reagents.

In some instances, where a volume of reagent is dispensed to the substrate at a first location, and thereafter travels to a second location different from the first location, the volume of reagent may travel in a path or paths, such that the travel path or paths are coated with the reagent. In some cases, such travel path or paths may encompass a desired surface area (e.g., entire surface area, partial surface area(s), etc.) of the substrate. In some instances, two or more reagents may be mixed on the surface of the substrate, such as by being dispensed at the same location and/or by directing a first reagent to travel to meet additional reagent(s). In some instances, the mixture of reagents formed on the substrate may be homogenous or substantially homogenous. The mixture of reagents may be formed at a first location on the substrate prior to dispersing the mixing of reagents to other locations on the substrate, such as at locations to meet other reagents or analytes.

In some embodiments, one or more solutions may be delivered directly to the reaction site without substantial displacement of the one or more solution from the point of delivery. Methods of direct delivery of a solution to the reaction site may include aerosol delivery of the solution, applying the solution using an applicator, curtain-coating the solution, slot-die coating, dispensing the solution from a translating dispense probe, dispensing the solution from an array of dispense probes, dipping the substrate into the solution, or contacting the substrate to a sheet comprising the solution.

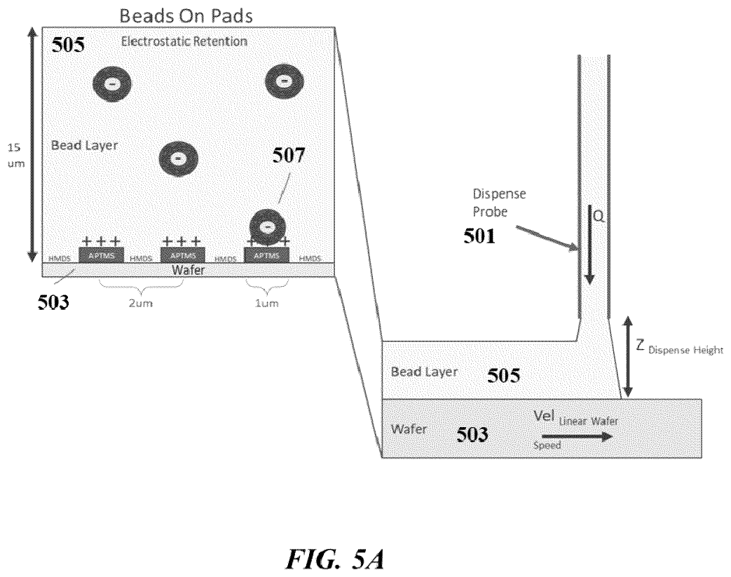

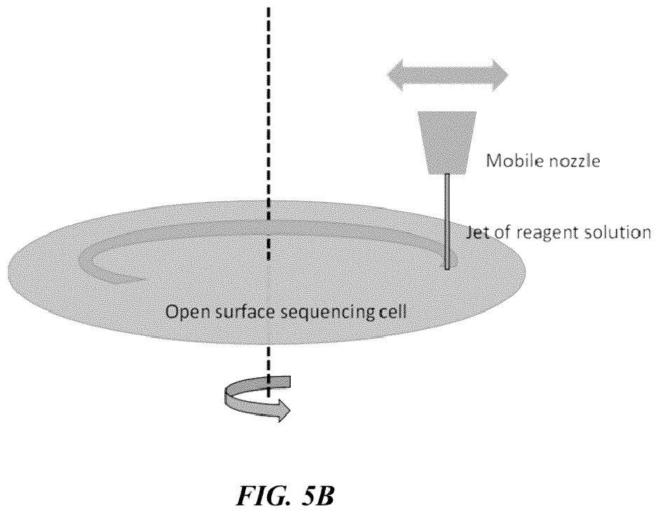

Aerosol delivery may comprise delivering a solution to the substrate in aerosol form by directing the solution to the substrate using a pressure nozzle or an ultrasonic nozzle. Applying the solution using an applicator may comprise contacting the substrate with an applicator comprising the solution and translating the applicator relative to the substrate. For example, applying the solution using an applicator may comprise painting the substrate. The solution may be applied in a pattern by translating the applicator, rotating the substrate, translating the substrate, or a combination thereof. Curtain-coating may comprise dispensing the solution from a dispense probe to the substrate in a continuous stream (e.g., a curtain or a flat sheet) and translating the dispense probe relative to the substrate. A solution may be curtain-coated in a pattern by translating the dispense probe, rotating the substrate, translating the substrate, or a combination thereof. Slot-die coating may comprise dispensing the solution from a dispense probe positioned near the substrate such that the solution forms a meniscus between the substrate and the dispense probe and translating the dispense probe relative to the substrate. A solution may be slot-die coated in a pattern by translating the dispense probe, rotating the substrate, translating the substrate, or a combination thereof. Dispensing the solution from a translating dispense probe may comprise translating the dispense probe relative to the substrate in a pattern (e.g., a spiral pattern, a circular pattern, a linear pattern, a striped pattern, a cross-hatched pattern, or a diagonal pattern). Dispensing the solution from an array of dispense probes may comprise dispensing the solution from an array of nozzles (e.g., a shower head) positioned above the substrate such that the solution is dispensed across an area of the substrate substantially simultaneously. Dipping the substrate into the solution may comprise dipping the substrate into a reservoir comprising the solution. In some embodiments, the reservoir may be a shallow reservoir to reduce the volume of the solution required to coat the substrate. Contacting the substrate to a sheet comprising the solution may comprise bringing the substrate in contact with a sheet of material (e.g., a porous sheet or a fibrous sheet) permeated with the solution. The solution may be transferred to the substrate. In some embodiments, the sheet of material may be a single-use sheet. In some embodiments, the sheet of material may be a reusable sheet. In some embodiments, a solution may be dispensed onto a substrate using the method illustrated in FIG. 5B, where a jet of a solution may be dispensed from a nozzle to a rotating substrate. The nozzle may translate radially relative to the rotating substrate, thereby dispensing the solution in a spiral pattern onto the substrate.

One or more solutions or reagents may be delivered to a substrate by any of the delivery methods disclosed herein. In some embodiments, two or more solutions or reagents are delivered to the substrate using the same or different delivery methods. In some embodiments, two or more solutions are delivered to the substrate such that the time between contacting a solution or reagent and a subsequent solution or reagent is substantially similar for each region of the substrate contacted to the one or more solutions or reagents. In some embodiments, a solution or reagent may be delivered as a single mixture. In some embodiments, the solution or reagent may be dispensed in two or more component solutions. For example, each component of the two or more component solutions may be dispensed from a distinct nozzle. The distinct nozzles may dispense the two or more component solutions substantially simultaneously to substantially the same region of the substrate such that a homogenous solution forms on the substrate. In some embodiments, dispensing of each component of the two or more components may be temporally separated. Dispensing of each component may be performed using the same or different delivery methods. In some embodiments, direct delivery of a solution or reagent may be combined with spin-coating.

A solution may be incubated on the substrate for any desired duration (e.g., minutes, hours, etc.). In some embodiments, the solution may be incubated on the substrate under conditions that maintain a layer of fluid on the surface. One or more of the temperature of the chamber, the humidity of the chamber, the rotation of the substrate, or the composition of the fluid may be adjusted such that the layer of fluid is maintained during incubation. In some instances, during incubation, the substrate may be rotated at an rotational frequency of no more than 60 rpm, 50 rpm, 40 rpm, 30 rpm, 25 rpm, 20 rpm, 15 rpm, 14 rpm, 13 rpm, 12 rpm, 11 rpm, 10 rpm, 9 rpm, 8 rpm, 7 rpm, 6 rpm, 5 rpm, 4 rpm, 3 rpm, 2 rpm, 1 rpm or less. In some cases, the substrate may be rotating with a rotational frequency of about 5 rpm during incubation.

The substrate or a surface thereof may comprise other features that aid in solution or reagent retention on the substrate or thickness uniformity of the solution or reagent on the substrate. In some cases, the surface may comprise a raised edge (e.g., a rim) which may be used to retain solution on the surface. The surface may comprise a rim near the outer edge of the surface, thereby reducing the amount of the solution that flows over the outer edge.

The dispensed solution may comprise any sample or any analyte disclosed herein. The dispensed solution may comprise any reagent disclosed herein. In some cases, the solution may be a reaction mixture comprising a variety of components. In some cases, the solution may be a component of a final mixture (e.g., to be mixed after dispensing). In non-limiting examples, the solution can comprise samples, analytes, supports, beads, probes, nucleotides, oligonucleotides, labels (e.g., dyes), terminators (e.g., blocking groups), other components to aid, accelerate, or decelerate a reaction (e.g., enzymes, catalysts, buffers, saline solutions, chelating agents, reducing agents, other agents, etc.), washing solution, cleavage agents, combinations thereof, deionized water, and other reagents and buffers.

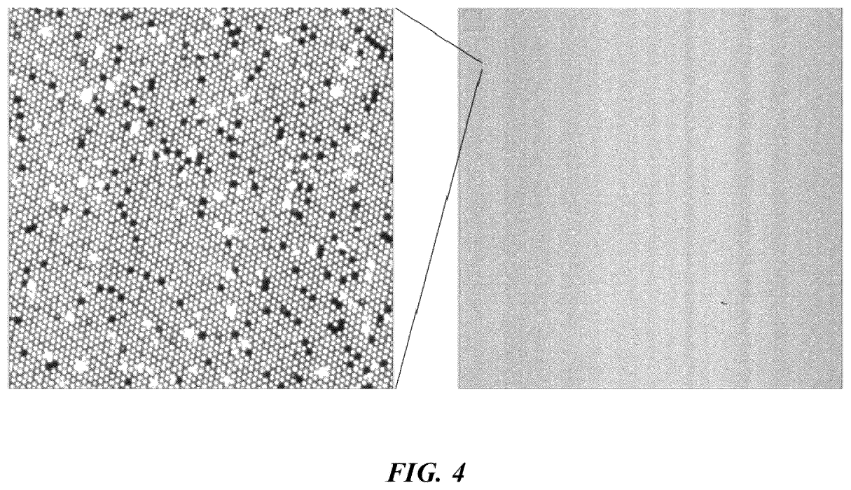

In some cases, a sample may be diluted such that the approximate occupancy of the individually addressable locations is controlled. In some cases, a sample may comprise beads, as described elsewhere herein, for example beads comprising nucleic acid colonies bound thereto. In some cases, an order of magnitude of at least about 10, 100, 1000, 10,000, 100,000, 1,000,000, 10,000,000, 100,000,000, 1,000,000,000, 10,000,000,000, 100,000,000,000 or more beads may be loaded on the substrate, such as to immobilize to as many individually addressable locations. Alternatively or in addition, an order of magnitude of at most about 100,000,000,000, 10,000,000,000, 1,000,000,000, 100,000,000, 10,000,000, 1,000,000, 100,000, 10,000,1000, 100, or 10 beads may be loaded on the substrate, such as to immobilize to as many individually addressable locations. In some cases, the beads may be distinguishable from one another using a property of the beads, such as color, reflectance, anisotropy, brightness, fluorescence, etc. In some cases, as described elsewhere herein, different beads may comprise different tags (e.g., nucleic acid sequences) coupled thereto. For example, a bead may comprise an oligonucleotide molecule comprising a tag that identifies a bead amongst a plurality of beads. FIG. 4 illustrates images of a portion of a substrate surface after loading a sample containing beads onto a substrate patterned with a substantially hexagonal lattice of individually addressable locations, where the right panel illustrates a zoomed-out image of a portion of a surface, and the left panel illustrates a zoomed-in image of a section of the portion of the surface. In some cases, after sample loading, a “bead occupancy” may generally refer to the number of individually addressable locations of a type comprising at least one bead out of the total number of individually addressable locations of the same type. A bead “landing efficiency” may generally refer to the number of beads that bind to the surface out of the total number of beads dispensed on the surface.

In some cases, beads may be dispensed to the substrate according to one or more systems and methods shown in FIGS. 5A-5B. As shown in FIG. 5A, a solution comprising beads may be dispensed from a dispense probe 501 (e.g., a nozzle) to a substrate 503 (e.g., a wafer) to form a layer 505. The dispense probe may be positioned at a height (“Z”) above the substrate. In the illustrated example, the beads are retained in the layer 505 by electrostatic retention, and may immobilize to the substrate at respective individually addressable locations. A set of beads in the solution may each comprise a population of amplified products (e.g., nucleic acid molecules) immobilized thereto, which amplified products accumulate to a negative charge on the bead with affinity to a positive charge. Otherwise, the beads may comprise reagents that have a negative charge. The substrate comprises alternating surface chemistry between distinguishable locations, in which a first location type comprises APTMS carrying a positive charge with affinity towards the negative charge of the amplified bead (e.g., a bead comprising amplified products immobilized thereto, and as distinguished from a negative bead which does not the comprise the same) or other bead comprising the negative charge, and a second location type comprises HMDS which has lower affinity and/or is repellant of the amplified bead or other bead comprising the negative charge. Within the layer 505 a bead may successfully land on a first location of the first location type (as in 507). In the illustrated example, the location size is 1 micron, the pitch between the different locations of the same location type (e.g., first location type) is 2 microns, and the layer has a depth of 15 micron. FIG. 5B illustrates a reagent (e.g., beads) being dispensed along a path on an open surface of the substrate. As shown in FIG. 5B, a reagent solution may be dispensed from a dispense probe (e.g., a nozzle). The reagent may be dispensed on the surface in any desired pattern or path. This may be achieved by moving one or both of the substrate and the dispense nozzle. The substrate and the dispense probe may move in any configuration with respect to each other to achieve any pattern (e.g., linear pattern, substantially spiral pattern, etc.).

In some instances, a subset or an entirety of the solution(s) may be recycled after the solution(s) have contacted the substrate. Recycling may comprise collecting, filtering, and reusing the subset or entirety of the solution. The filtering may be molecule filtering.

### Detection

An optical system comprising a detector may be configured to detect one or more signals from a detection area on the substrate prior to, during, or subsequent to, the dispensing of reagents to generate an output. Signals from multiple individually addressable locations may be detected during a single detection event. Signals from the same individually addressable location may be detected in multiple instances.

A detectable signal, such as an optical signal (e.g., fluorescent signal), may be generated upon a reaction between a probe in the solution and the analyte. For example, the signal may originate from the probe and/or the analyte. The detectable signal may be indicative of a reaction or interaction between the probe and the analyte. The detectable signal may be a non-optical signal. For example, the detectable signal may be an electronic signal. The detectable signal may be detected by a detector (e.g., one or more sensors). For example, an optical signal may be detected via one or more optical detectors in an optical detection scheme described elsewhere herein. The signal may be detected during rotation of the substrate. The signal may be detected following termination of the rotation. The signal may be detected while the analyte is in fluid contact with a solution. The signal may be detected following washing of the solution. In some instances, after the detection, the signal may be muted, such as by cleaving a label from the probe and/or the analyte, and/or modifying the probe and/or the analyte. Such cleaving and/or modification may be affected by one or more stimuli, such as exposure to a chemical, an enzyme, light (e.g., ultraviolet light), or temperature change (e.g., heat). In some instances, the signal may otherwise become undetectable by deactivating or changing the mode (e.g., detection wavelength) of the one or more sensors, or terminating or reversing an excitation of the signal. In some instances, detection of a signal may comprise capturing an image or generating a digital output (e.g., between different images).

The operations of (i) directing a solution to the substrate and (ii) detection of one or more signals indicative of a reaction between a probe in the solution and an analyte immobilized to the substrate, may be repeated any number of times. Such operations may be repeated in an iterative manner. For example, the same analyte immobilized to a given location in the array may interact with multiple solutions in the multiple repetition cycles. For each iteration, the additional signals detected may provide incremental, or final, data about the analyte during the processing. For example, where the analyte is a nucleic acid molecule and the processing is sequencing, additional signals detected for each iteration may be indicative of a base in the nucleic acid sequence of the nucleic acid molecule. In some cases, multiple solutions can be provided to the substrate without intervening detection events. In some cases, multiple detection events can be performed after a single flow of solution. In some instances, a washing solution, cleaving solution (e.g., comprising cleavage agent), and/or other solutions may be directed to the substrate between each operation, between each cycle, or a certain number of times for each cycle.

The optical system may be configured for continuous area scanning of a substrate during rotational motion of the substrate. The term “continuous area scanning (CAS),” as used herein, generally refers to a method in which an object in relative motion is imaged by repeatedly, electronically or computationally, advancing (clocking or triggering) an array sensor at a velocity that compensates for object motion in the detection plane (focal plane). CAS can produce images having a scan dimension larger than the field of the optical system. TDI scanning may be an example of CAS in which the clocking entails shifting photoelectric charge on an area sensor during signal integration. For a TDI sensor, at each clocking step, charge may be shifted by one row, with the last row being read out and digitized. Other modalities may accomplish similar function by high speed area imaging and co-addition of digital data to synthesize a continuous or stepwise continuous scan.

The optical system may comprise one or more sensors. The sensors may detect an image optically projected from the sample. The optical system may comprise one or more optical elements. An optical element may be, for example, a lens, prism, mirror, wave plate, filter, attenuator, grating, diaphragm, beam splitter, diffuser, polarizer, depolarizer, retroreflector, spatial light modulator, or any other optical element. The system may comprise any number of sensors. In some cases, a sensor is any detector as described herein. In some examples, the sensor may comprise image sensors, CCD cameras, CMOS cameras, TDI cameras (e.g., TDI line-scan cameras), pseudo-TDI rapid frame rate sensors, or CMOS TDI or hybrid cameras. The optical system may further comprise any optical source. In some cases, where there are multiple sensors, the different sensors may image the same or different regions of the rotating substrate, in some cases simultaneously. Each sensor of the plurality of sensors may be clocked at a rate appropriate for the region of the rotating substrate imaged by the sensor, which may be based on the distance of the region from the center of the rotating substrate or the tangential velocity of the region. In some cases, multiple scan heads can be operated in parallel along different imaging paths (e.g., interleaved spiral scans, nested spiral scans, interleaved ring scans, nested ring scans). A scan head may comprise one or more of a detector element such as a camera (e.g., a TDI line-scan camera), an illumination source (e.g., as described herein), and one or more optical elements (e.g., as described herein).

The system may further comprise a controller. The controller may be operatively coupled to the one or more sensors. The controller may be programmed to process optical signals from each region of the rotating substrate. For instance, the controller may be programmed to process optical signals from each region with independent clocking during the rotational motion. The independent clocking may be based at least in part on a distance of each region from a projection of the axis and/or a tangential velocity of the rotational motion. The independent clocking may be based at least in part on the angular velocity of the rotational motion. While a single controller has been described, a plurality of controllers may be configured to, individually or collectively, perform the operations described herein.

In some cases, the optical system may comprise an immersion objective lens. The immersion objective lens may be in contact with an immersion fluid that is in contact with the open substrate. The immersion fluid may comprise any suitable immersion medium for imaging (e.g., water, aqueous, organic solution). In some cases, an enclosure may partially or completely surround a sample-facing end of the optical imaging objective. The enclosure may be configured to contain the fluid. The enclosure may not be in contact with the substrate; for example, a gap between the enclosure and the substrate may be filled by the fluid contained by the enclosure (e.g., the enclosure can retain the fluid via surface tension). In some cases, an electric field may be used to regulate a hydrophobicity of one or more surfaces of the container to retain at least a portion of the fluid contacting the immersion objective lens and the open substrate

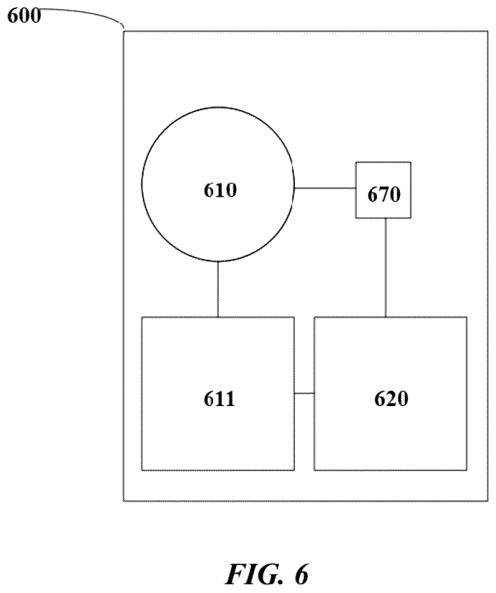

FIG. 6 shows a computerized system 600 for sequencing a nucleic acid molecule. The system may comprise a substrate 610, such as any substrate described herein. The system may further comprise a fluid flow unit 611. The fluid flow unit may comprise any element associated with fluid flow described herein. The fluid flow unit may be configured to direct a solution comprising a plurality of nucleotides described herein to an array of the substrate prior to or during rotation of the substrate. The fluid flow unit may be configured to direct a washing solution described herein to an array of the substrate prior to or during rotation of the substrate. In some instances, the fluid flow unit may comprise pumps, compressors, and/or actuators to direct fluid flow from a first location to a second location. The fluid flow unit may be configured to direct any solution to the substrate 610. The fluid flow system may be configured to collect any solution from the substrate 610. The system may further comprise a detector 670, such as any detector described herein. The detector may be in sensing communication with the substrate surface.

The system may further comprise one or more processors 620. The one or more processors may be individually or collectively programmed to implement any of the methods described herein. For instance, the one or more processors may be individually or collectively programmed to implement any or all operations of the methods of the present disclosure. In particular, the one or more processors may be individually or collectively programmed to: (i) direct the fluid flow unit to direct the solution comprising the plurality of nucleotides across the array during or prior to rotation of the substrate; (ii) subject the nucleic acid molecule to a primer extension reaction under conditions sufficient to incorporate at least one nucleotide from the plurality of nucleotides into a growing strand that is complementary to the nucleic acid molecule; and (iii) use the detector to detect a signal indicative of incorporation of the at least one nucleotide, thereby sequencing the nucleic acid molecule.

### High Throughput

An open substrate system of the present disclosure may comprise a barrier system configured to maintain a fluid barrier between a sample processing environment and an exterior environment. The barrier system is described in further detail in International Pub. No. WO2020/118172, which is entirely incorporated herein by reference. A sample environment system may comprise a sample processing environment defined by a chamber and a lid plate, where the lid plate is not in contact with the chamber. The gap between the lid plate and the chamber may comprise the fluid barrier. The fluid barrier may comprise fluid (e.g., air) from the sample processing environment and/or the exterior environment and may have lower pressure than the sample environment, the external environment, or both. The fluid in the fluid barrier may be in coherent motion or bulk motion.

The sample processing environment may comprise therein a substrate, such as any substrate described elsewhere herein. Any operation performed on or with the substrate, as described elsewhere herein, may be performed within the sample processing environment while the fluid barrier is maintained. For example, the substrate may be rotated within the sample processing environment during various operations. In another example, fluid may be directed to the substrate while the substrate is in the sample processing environment, via a fluid handler (e.g., nozzle) that penetrates the lid plate into the sample processing environment. In another example, a detector can image the substrate while the substrate is in the sample processing environment, via a detector that penetrates the lid plate into the sample processing environment. Beneficially, the fluid barrier may help maintain temperature(s) and/or relative humidit(ies), or ranges thereof, within the sample processing environment during various processing operations.

The systems described herein, or any element thereof, may be environmentally controlled. For instance, the systems may be maintained at a specified temperature or humidity. For an operation, the systems (or any element thereof) may be maintained at a temperature of at least 20 degrees Celsius (° C.), 25° C., 30° C., 35° C., 40° C., 45° C., 50° C., 55° C., 60° C., 65° C., 70° C., 75° C., 80° C., 85° C., 90° C., 95° C., 100° C., or more. Alternatively or in addition, for an operation, the systems (or any element thereof) may be maintained at a temperature of at most 100° C., 95° C., 90° C., 85° C., 80° C., 75° C., 70° C., 65° C., 60° C., 55° C., 50° C., 45° C., 40° C., 35° C., 30° C., 25° C., 20° C., or less. Different elements of the system may be maintained at different temperatures or within different temperature ranges, such as the temperatures or temperature ranges described herein. Elements of the system may be set at temperatures above the dew point to prevent condensation. Elements of the system may be set at temperatures below the dew point to collect condensation. In one example, a sample processing environment comprising a substrate as described elsewhere herein may be environmentally controlled from an exterior environment. The sample processing environment may be further divided into separate regions which are maintained at different local temperatures and/or relative humidities, such as a first region contacting or in proximity to a surface of the substrate, and a second region contacting or in proximity to a top portion of the sample processing environment (e.g., a lid). For example, the local environment of the first region may be maintained at a first set of temperatures and first set of humidities configured to prevent or minimize evaporation of one or more reagents on the surface of the substrate, and the local environment of the second region may be maintained at a second set of temperatures and second set of humidities configured to enhance or restrict condensation. The first set of temperatures may be the lowest temperatures within the sample processing environment and the second set temperatures may be the highest temperatures within the sample processing environment.

In some instances, the environmental conditions of the different regions may be achieved by controlling the temperature of the enclosure. In some instances, the environmental conditions of the different regions may be achieved by controlling the temperature of selected parts or whole of the container. In some instances, the environmental conditions of the different regions may be achieved by controlling the temperature of selected parts or whole of the substrate. In some instances, the environmental conditions of the different regions may be achieved by controlling the temperature of reagents dispensed to the substrate. Any combination thereof may be used to control the environmental conditions of the different regions. Heat transfer may be achieved by any method, including for example, conductive, convective, and radiative methods.

While examples described herein provide relative rotational motion of the substrates and/or detector systems, the substrates and/or detector systems may alternatively or additionally undergo relative non-rotational motion, such as relative linear motion, relative non-linear motion (e.g., curved, arcuate, angled, etc.), and any other types of relative motion.

In some instances, an open substrate is retained in the same or approximately the same physical location during processing of an analyte and subsequent detection of a signal associated with a processed analyte.

In some instances, different operations on or with the open substrate are performed in different stations. Different stations may be disposed in different physical locations. For example, a first station may be disposed above, below, adjacent to, or across from a second station. In some cases, the different stations can be housed within an integrated housing. Alternatively, the different stations can be housed separately. In some cases, different stations may be separated by a barrier, such as a retractable barrier (e.g., sliding door). One or more different stations of a system, or portions thereof, may be subjected to different physical conditions, such as different temperatures, pressures, or atmospheric compositions. In an example, a processing station may comprise a first atmosphere comprising a first set of conditions and a second atmosphere comprising a second set of conditions. The barrier systems may be used to maintain different physical conditions of one or more different stations of the system, or portions thereof, as described elsewhere herein.

The open substrate may transition between different stations by transporting a sample processing environment containing the open substrate (such as the one described with respect to the barrier system) between the different stations. One or more mechanical components or mechanisms, such as a robotic arm, elevator mechanism, actuators, rails, and the like, or other mechanisms may be used to transport the sample processing environment.

An environmental unit (e.g., humidifiers, heaters, heat exchangers, compressors, etc.) may be configured to regulate one or more operating conditions in each station. In some instances, each station may be regulated by independent environmental units. In some instances, a single environmental unit may regulate a plurality of stations. In some instances, a plurality of environmental units may, individually or collectively, regulate the different stations. An environmental unit may use active methods or passive methods to regulate the operating conditions. For example, the temperature may be controlled using heating or cooling elements. The humidity may be controlled using humidifiers or dehumidifiers. In some instances, a part of a particular station, such as within a sample processing environment, may be further controlled from other parts of the particular station. Different parts may have different local temperatures, pressures, and/or humidity.

In one example, the delivery and/or dispersal of reagents may be performed in a first station having a first operating condition, and the detection process may be performed in a second station having a second operating condition different from the first operating condition. The first station may be at a first physical location in which the open substrate is accessible to a fluid handling unit during the delivery and/or dispersal processes, and the second station may be at a second physical location in which the open substrate is accessible to the detector system.

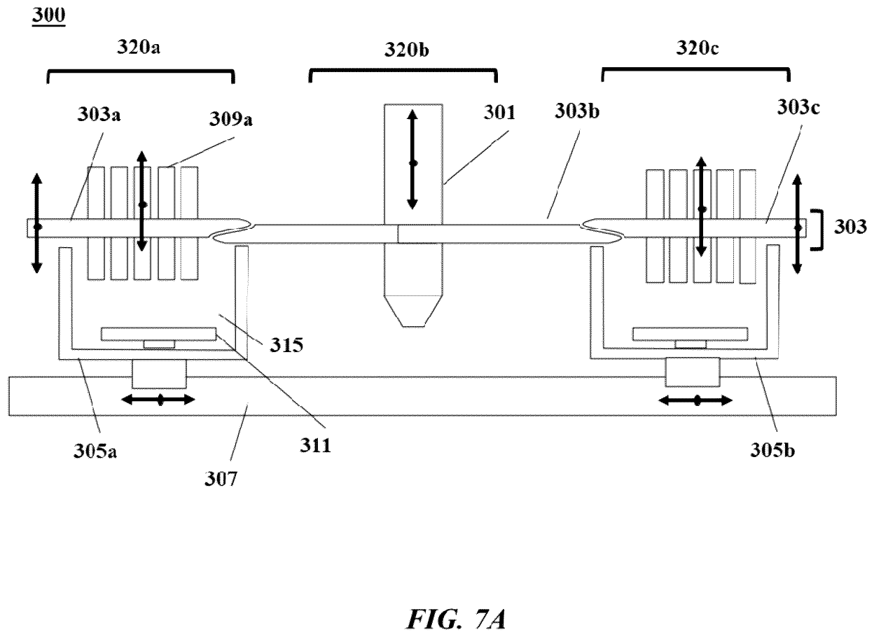

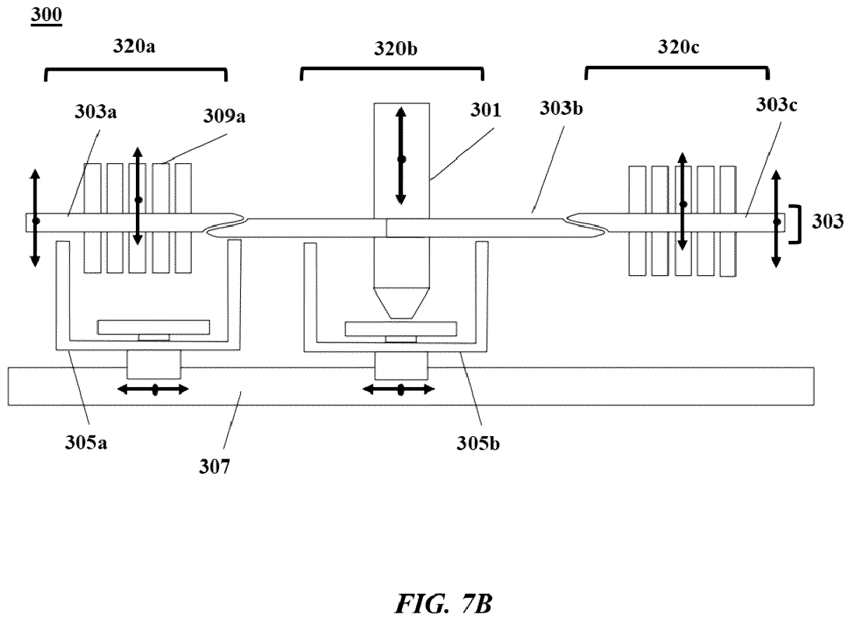

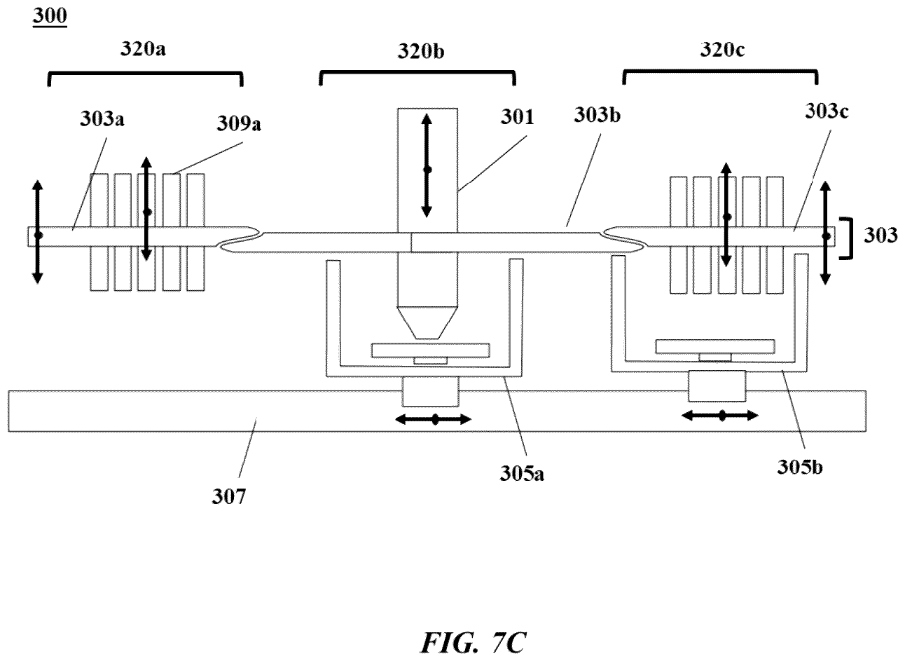

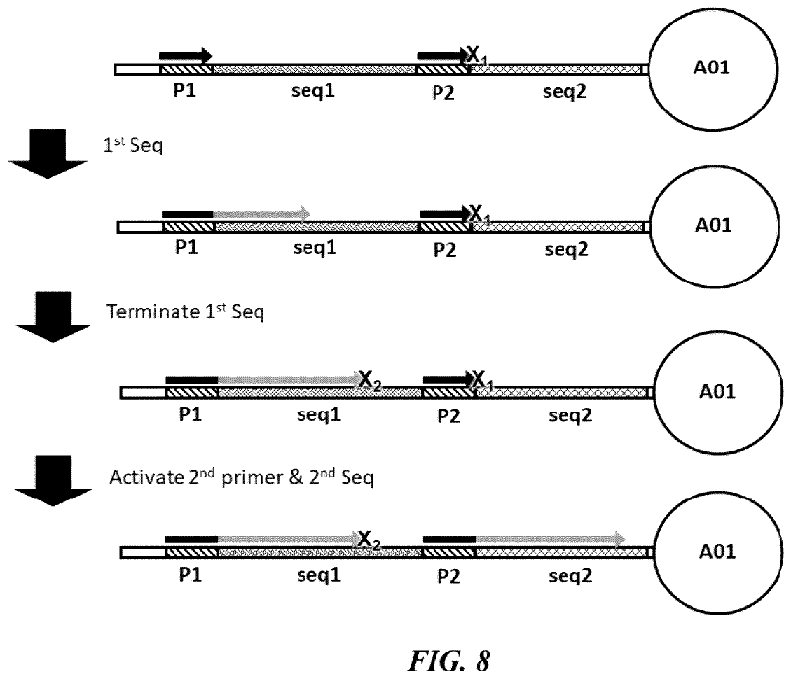

One or more modular sample environment systems (each having its own barrier system) can be used between the different stations. In some instances, the systems described herein may be scaled up to include two or more of a same station type. For example, a sequencing system may include multiple processing and/or detection stations. FIGS. 7A-7C illustrate a system 300 that multiplexes two modular sample environment systems in a three-station system. In FIG. 7B, a first chemistry station (e.g., 320a) can operate (e.g., dispense reagents, e.g., to incorporate nucleotides to perform sequencing by synthesis) via at least a first operating unit (e.g., fluid dispenser 309a) on a first substrate (e.g., 311) in a first sample environment system (e.g., 305a) while substantially simultaneously, a detection station (e.g., 320b) can operate (e.g., scan) on a second substrate in a second sample environment system (e.g., 305b) via at least a second operating unit (e.g., detector 301), while substantially simultaneously, a second chemistry station (e.g., 320c) sits idle. An idle station may not operate on a substrate. An idle station (e.g., 320c) may be recharged, reloaded, replaced, cleaned, washed (e.g., to flush reagents), calibrated, reset, kept active (e.g., power on), and/or otherwise maintained during an idle time. After an operating cycle is complete, the sample environment systems may be re-stationed, as in FIG. 7C, where the second substrate in the second sample environment system (e.g., 305b) is re-stationed from the detection station (e.g., 320b) to the second chemistry station (e.g., 320c) for operation (e.g., dispensing of reagents, e.g., to incorporate nucleotides to perform sequencing by synthesis) by the second chemistry station, and the first substrate in the first sample environment system (e.g., 305a) is re-stationed from the first chemistry station (e.g., 320a) to the detection station (e.g., 320b) for operation (e.g., scanning) by the detection station. An operating cycle may be deemed complete when operation at each active, parallel station is complete. During re-stationing, the different sample environment systems may be physically moved (e.g., along the same track or dedicated tracks, e.g., rail(s) 307) to the different stations and/or the different stations may be physically moved to the different sample environment systems. One or more components of a station, such as modular plates 303a, 303b, 303c of plate 303 defining a particular station(s), may be physically moved to allow a sample environment system to exit the station, enter the station, or cross through the station. During processing of a substrate at station, the environment of a sample environment region (e.g., 315) of a sample environment system (e.g., 305a) may be controlled and/or regulated according to the station's requirements. After the next operating cycle is complete, the sample environment systems can be re-stationed again, such as back to the configuration of FIG. 7B, and this re-stationing can be repeated (e.g., between the configurations of FIGS. 7B and 7C) with each completion of an operating cycle until the required processing for a substrate is completed. In this illustrative re-stationing scheme, the detection station may be kept active (e.g., not have idle time not operating on a substrate) for all operating cycles by providing alternating different sample environment systems to the detection station for each consecutive operating cycle. Beneficially, use of the detection station is optimized. Based on different processing or equipment needs, an operator may opt to run the two chemistry stations (e.g., 320a, 320c) substantially simultaneously while the detection station (e.g., 320b) is kept idle, such as illustrated in FIG. 7A.

Beneficially, different operations within the system may be multiplexed with high flexibility and control. For example, as described herein, one or more processing stations may be operated in parallel with one or more detection stations on different substrates in different modular sample environment systems to reduce or eliminate lag between different sequences of operations (e.g., chemistry first, then detection). The modular sample environment systems may be translated between the different stations accordingly to optimize efficient equipment use (e.g., such that the detection station is in operation almost 100% of the time). In some examples, at least 1, 2, 3, 4, 5, 6, 7, 8, 9, or more modules or stations of the sequencing system may be multiplexed. For example, 2 or more of the modules may each perform their intended function simultaneously or according to the methods described elsewhere herein. An example of this may comprise two-station multiplexing of an optics station and a chemistry station as described herein. Another example may comprise multiplexing three or more stations and process phases. For example, the method may comprise using staggered chemistry phases sharing a scanning station. The scanning station may be a high-speed scanning station. The modules or stations may be multiplexed using various sequences and configurations.

The nucleic acid sequencing systems and optical systems described herein (or any elements thereof) may be combined in a variety of architectures.

Sequencing with Multi-Priming

Provided herein are devices, systems, methods, compositions, and kits for sequencing with multi-priming. Such devices, systems, methods, compositions, and kits can be applied alternatively or in addition to the sequencing operation 107 described with respect to sequencing workflow 100 of FIG. 1. Such devices, systems, methods, compositions, and kits can be used in conjunction with the sample processing systems and methods, or components thereof (e.g., substrates, detectors, reagent dispensing, continuous scanning, etc.) described herein.

The devices, systems, methods, compositions, and kits provided herein may allow for the selective sequencing of multiple, distinct regions of a template nucleic acid, and/or allow to selectively not sequence one more distinct regions of the template nucleic acid.

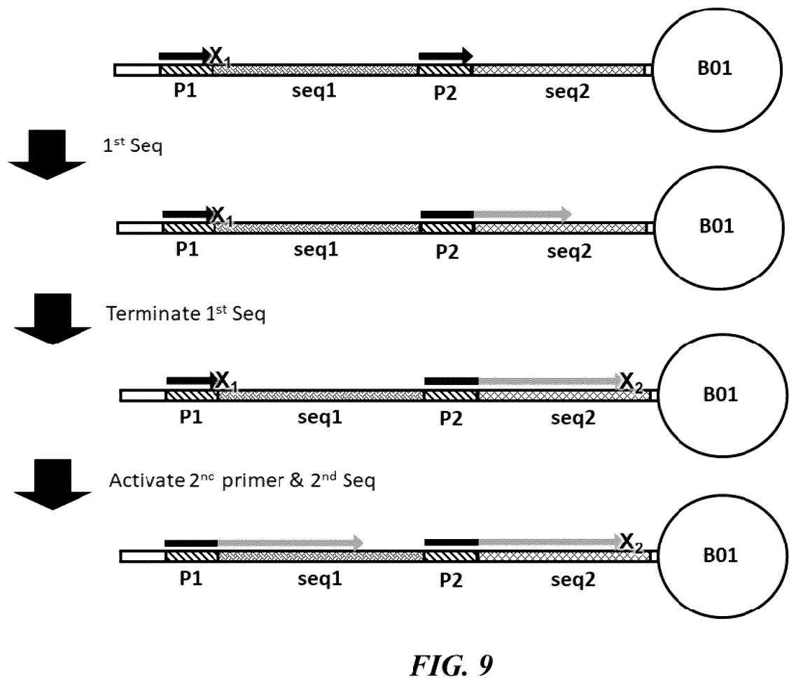

In some cases, the sequencing operation (e.g., 107) may comprise providing multiple, distinct sequencing primer molecules to a template nucleic acid. Example schemes are illustrated in FIGS. 8-9.

Referring to FIG. 8, a bead A01 may comprise a template nucleic acid coupled thereto. The template nucleic acid illustrated in FIG. 8 may be one of many copies (others not illustrated) coupled to the same bead. A colony of template nucleic acids may beneficially amplify sequencing signals interrogated during or between sequencing flows. Multiple primer molecules may be provided to bind to different regions of the template nucleic acid, such as a first primer molecule that binds to a first target region (“P1”) and a second primer molecule that binds to a second target region (“P2”), where the first target region is disposed 3′ to the second target region. The second primer molecule may be inactivated for extension. For example, the second primer molecule may be protected by a blocking moiety (e.g., “X₁”), such as a dideoxynucleotide, to prevent extension. The sequencing reaction space may be subjected to conditions sufficient to sequence a first region (“seq1”) of the template nucleic acid from the first target region via extending the first primer molecule. Various sequencing flows that can be used, e.g., SBS flows, are described elsewhere herein. The first region sequencing may terminate. In some instances, the termination occurs via incorporation of a blocking moiety (e.g., “X₂”). The second primer molecule may be activated for extension. For example, the blocking moiety (“X₁”) of the second primer molecule may be removed, e.g., cleaved off Various cleavage mechanisms are described elsewhere herein. Then, the sequencing reaction space may be subjected to conditions sufficient to sequence a second region (“seq2”) of the template nucleic acid from the second target region via extending the second primer molecule. The second region sequencing may terminate. In some cases, the first region and the second region that are sequenced may be adjacent on the template nucleic acid. In some cases, the first region and the second region that are sequenced may be non-adjacent on the template nucleic acid. For example, there may be an intermediary sequence between the two sequenced regions. The respective sequencing signals collected from a sequencing reaction space during the two sequencing workflows may be related back to each other, such as to associate them to the same bead A01 and same template nucleic acid, based on a location of the sequencing signals collected. For example, the bead A01 may be immobilized to an individually addressable location of a substrate during sequencing, as described elsewhere herein, and sequencing signals associated with the individually addressable location may be associated with the same bead and same template nucleic acid.

Referring to FIG. 9, a similar workflow as the one in FIG. 8 is illustrated except that instead of the second primer molecule, the first primer molecule is inactivated for extension. Multiple primer molecules may be provided to bind to different regions of the template nucleic acid coupled to bead B01, such as a first primer molecule that binds to a first target region (“P1”) and a second primer molecule that binds to a second target region (“P2”), where the first target region is disposed 3′ to the second target region. The first primer molecule may be inactivated for extension, e.g., by a blocking moiety (e.g., “X₁), to prevent extension. The sequencing reaction space may be subjected to conditions sufficient to sequence a second region (“seq2”) of the template nucleic acid from the second target region via extending the second primer molecule. The second region sequencing may terminate. In some instances, the termination occurs via incorporation of a blocking moiety (e.g., “X₂”). The first primer molecule may be activated for extension. For example, the blocking moiety (“X₁”) of the first primer molecule may be removed, e.g., cleaved off. Then, the sequencing reaction space may be subjected to conditions sufficient to sequence a first region (“seq1”) of the template nucleic acid from the first target region via extending the first primer molecule. The first region sequencing may terminate.

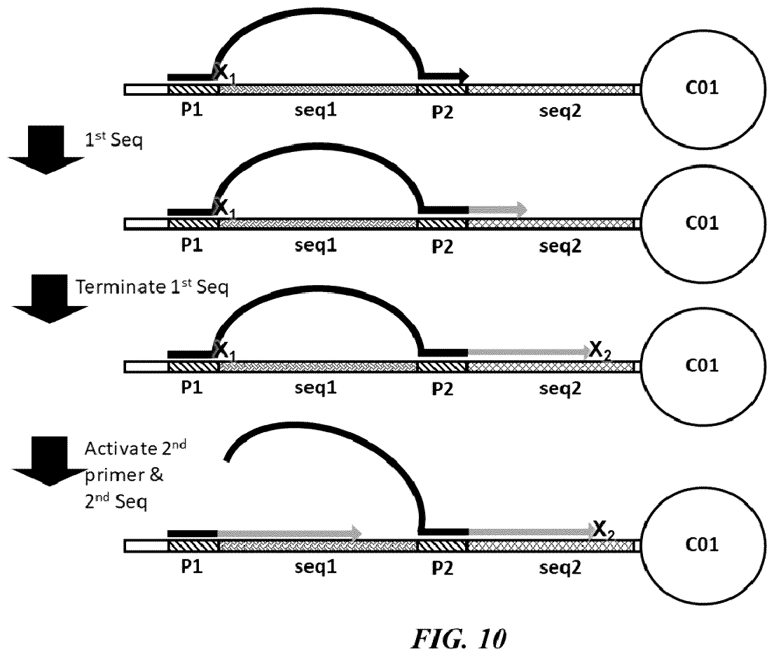

In some cases, the sequencing operation (e.g., 107) may comprise providing a connected sequencing primer molecule to a template nucleic acid, the connected sequencing primer molecule comprising multiple priming regions. An example scheme is illustrated in FIG. 10.

Referring to FIG. 10, a bead C01 may comprise a template nucleic acid coupled thereto. The template nucleic acid illustrated in FIG. 10 may be one of many copies (others not illustrated) coupled to the same bead. A connected primer molecule comprising (i) a first primer region, (ii) a connector region, and (iii) a second primer region may be provided to bind to different regions of the template nucleic acid. The first primer region may bind to a first target region (“P1”), a second primer region may bind to a second target region (“P2”), where the first target region is disposed 3′ to the second target region, with the connector region unbound to any portion of the template nucleic acid. The sequencing reaction space may be subjected to conditions sufficient to sequence a second region (“seq2”) of the template nucleic acid from the second target region via extending the connected primer molecule from the second primer region. Various sequencing flows that can be used, e.g., SBS flows, are described elsewhere herein. The second region sequencing may terminate. In some instances, the termination occurs via incorporation of a blocking moiety (e.g., “X₂”). The first primer region may be activated for extension, such as by cleaving the connected primer molecule. For example, a cleavage site (e.g., “X₁”) disposed between the first primer region and the connector region may be cleaved to open the first primer region for extension through a first region (“seq1”) of the template nucleic acid. Then, the sequencing reaction space may be subjected to conditions sufficient to sequence the first region (“seq1”) of the template nucleic acid from the first target region via extending from the first primer region. The first region sequencing may terminate. In some cases, the first region and the second region that are sequenced may be non-adjacent on the template nucleic acid. For example, there may be an intermediary sequence between the two sequenced regions. The respective sequencing signals collected from a sequencing reaction space during the two sequencing workflows may be related back to each other, such as to associate them to the same bead C01 and same template nucleic acid, based on a location of the sequencing signals collected. For example, the bead C01 may be immobilized to an individually addressable location of a substrate during sequencing, as described elsewhere herein, and sequencing signals associated with the individually addressable location may be associated with the same bead and same template nucleic acid.

Accordingly, a method for sequencing multiple regions in a nucleic acid molecule may comprise: (a) hybridizing a first primer molecule and a second primer molecule to a first target region and a second target region, respectively, of a target nucleic acid strand, wherein an intermediary region is disposed between the first target region and the second target region on the target nucleic acid strand, wherein the second primer molecule is inactivated for extension; (b) extending the first primer molecule to generate a first sequencing read for a first region of the target nucleic acid strand; (c) activating the second primer molecule for extension; and (d) extending the second primer molecule to generate a second sequencing read for a second region of the target nucleic acid strand, wherein the second region is different from the first region. Another method for sequencing multiple regions in a nucleic acid molecule may comprise: (a) hybridizing a first primer region and a second primer region of a primer molecule to a first target region and a second target region, respectively, of a target nucleic acid strand, wherein an intermediary region is disposed between the first target region and the second target region on the target nucleic acid strand, wherein a connector region is disposed between the first primer region and the second primer region; (b) extending the primer molecule from the second primer region to generate a first sequencing read for a first region of the target nucleic acid strand; (c) cleaving the primer molecule to activate the first primer region for extension; and (d) extending the primer molecule from the first primer region to generate a second sequencing read for a second region of the target nucleic acid strand, wherein the second region is different from the first region.

In these methods, as described with respect to FIGS. 8-9, the second primer molecule may initially be inactivated for extension, then activated for extension. The activation may be facilitated by cleavage of one or more cleavable or excisable moieties. In some cases, the second primer molecule may be inactivated by a blocking moiety (e.g., “X₁”), such as a dideoxynucleotide, which may be cleaved to activate the second primer molecule for extension. In some cases, the second primer molecule comprises at least one cleavable or excisable moiety at a cleavage site. In some cases, the second primer molecule comprises a plurality of cleavable or excisable moieties at a cleavage site. The cleavage site, for example, may be disposed 5′ of the blocking moiety. In these methods, as described with respect to FIG. 10, the first primer region may be activated for extension, such as by cleaving the connected primer molecule the first primer region. The connected primer molecule may comprise one or more cleavable or excisable moieties at a cleavage site.

As used herein, the term “cleavable or excisable moiety” generally refers to any moiety that may be cleaved and/or excised from a nucleic acid molecule. A cleavable or excisable moiety may be a cleavable base. As used herein, the term “cleavable base” generally refers to any base or analog of a base (e.g., nucleobase) that can be specifically cleaved and removed or excised from a nucleic acid molecule. Examples of cleavable bases include, but are not limited to, uracil, 8-oxoguanine (also referred to as 8-hydroxyguanine, 8-oxo-7,8-dihydroquinine, 7,8-dihydro-8-oxoguanine, and 8oxoG herein), inosine, an RNA base, and 2,6-diamino-4-hydroxy-5-formamidopyrimidine (FapyG). In some embodiments, the uracil is a DNA uracil base. In some embodiments, the RNA base is in a DNA backbone. In some embodiments, the DNA is devoid of RNA bases other than the cleavable or excisable bases. In some embodiments, the DNA backbone is devoid of RNA bases other than the cleavable or excisable bases. A cleavable or excisable moiety may comprise azobenzene. A cleavable or excisable moiety may comprise an abasic site. A cleavable or excisable moiety may be a photocleavable moiety. A cleavable or excisable moiety may be a C3 or other spacer.

Cleavage and/or excision of a cleavable or excisable moiety may be carried out by contacting the cleavable or excisable moiety (e.g., cleavable base) with a cleaving agent. Examples of cleaving agents include, but are not limited to, uracil DNA glycosylase (UDG), apyrimidinic/apurinic endonuclease (APE), endonucleases (e.g., endonuclease VIII (EndoVIII) or V (EndoV)), uracil-specific excision reagent (USER) enzyme, formamidopyrimidine DNA glycosylase (Fpg), 8-oxoguanine glycosylase (OGGI), and RNase (e.g., RNaseH, such as RNaseHII). Photocleavable or photoexcisable moieties may be cleaved or excised using appropriate application of energy, such as by contacting the moiety with ultraviolet (UV) light or radiation, or other photo stimulus. In some cases, the cleavage agent may comprise a dosage of radiation at a predetermined wavelength or wavelength range. For example, the radiation may be at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 nanometers (nm) or higher. Alternatively or in addition, the radiation may be at most about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 nanometers (nm) or lower. One or more cleaving agents may be used in combination to cleave or excise a cleavable or excisable moiety. In an example, the cleavable base may be an RNA base in a DNA backbone, and the cleaving agent may be RNase (e.g., RNaseH or RNaseHII). In such a case, the nucleic acid molecule may not be an RNA molecule. In such a case the nucleic acid molecule may be devoid of RNA bases other than the cleavable or excisable bases. In some embodiments, the first cleavable or excisable base is the only RNA base, the second cleavable or excisable base is the only RNA base, the third cleavable or excisable base is the only RNA base, or a combination thereof. In another example, the cleavable base may be a uracil base and the cleaving agent may be selected from uracil DNA glycosylase (UDG), apyrimidinic/apurinic endonuclease (APE), Endonuclease VIII and uracil-specific excision reagent (USER) enzyme. For example, the cleaving agent may be UDG. For example, the cleaving agent may be APE. For example, the cleaving agent may be USER enzyme. In another example, the cleavable base may be an inosine base and the cleaving agent may be Endonuclease V (Endo V). In another example, the cleavable base may be 2,6-diamino-4-hydroxy-5-formamidopyrimidine (FapyG) base and the cleaving agent may be formamidopyrimidine DNA glycosylase (Fpg). In another example, the cleavable base may be 8-oxo-7,8-dihydroquinine (8oxoG) and the cleaving agent may be 8-oxoguanine glycosylase (OGGI). In another example, the cleavable base may be a photo-cleavable base and the cleaving agent may be light, such as laser light. Application of a cleaving agent may generate a “nick” in a strand of a nucleic acid molecule. Alternatively, or in addition to, another enzyme may be added to generate a nick, or otherwise functionalize a nick. For example, T4 polynucleotide kinase may be added to remove a 3′ phosphate. An enzyme may be used to remove a lesion, such as a 3′ lesion.

A dose of radiation or UV radiation may be measured in joule per square centimeter (J/cm²; fluence). A dose of radiation or UV radiation may be at least about 1×10\hat{ }−15 J/cm², 1×10\hat{ }−14 J/cm², 1×10\hat{ }−13 J/cm², 1×10\hat{ }−12 J/cm², 1×10\hat{ }−11 J/cm², 1×10\hat{ }−10 J/cm², 1×10\hat{ }−9 J/cm², 1×10\hat{ }−8 J/cm², 1×10\hat{ }−7 J/cm², 1×10\hat{ }−6 J/cm², 1×10\hat{ }−5 J/cm², 1×10\hat{ }−4 J/cm², 1×10\hat{ }−3 J/cm², 1×10\hat{ }−2 J/cm², 1×10\hat{ }−1 J/cm², 1×10\hat{ }0 J/cm², 1×10\hat{ }1 J/cm², 1×10\hat{ }2 J/cm², 1×10\hat{ }3 J/cm², 1×10\hat{ }4 J/cm², 1×10\hat{ }5 J/cm², 1×10\hat{ }6 J/cm², 1×10\hat{ }7 J/cm², 1×10\hat{ }8 J/cm², 1×10\hat{ }9 J/cm² or more. A dose of radiation or UV radiation may be at most about 1×10\hat{ }−15 J/cm², 1×10\hat{ }−14 J/cm², 1×10\hat{ }−13 J/cm², 1×10\hat{ }−12 J/cm², 1×10\hat{ }−11 J/cm², 1×10\hat{ }−10 J/cm², 1×10\hat{ }−9 J/cm², 1×10\hat{ }−8 J/cm², 1×10\hat{ }−7 J/cm², 1×10\hat{ }−6 J/cm², 1×10\hat{ }−5 J/cm², 1×10\hat{ }−4 J/cm², 1×10\hat{ }−3 J/cm², 1×10\hat{ }−2 J/cm², 1×10\hat{ }−1 J/cm², 1×10\hat{ }0 J/cm², 1×10\hat{ }1 J/cm², 1×10\hat{ }2 J/cm², 1×10\hat{ }3 J/cm², 1×10\hat{ }4 J/cm², 1×10\hat{ }5 J/cm², 1×10\hat{ }6 J/cm², 1×10\hat{ }7 J/cm², 1×10\hat{ }8 J/cm², or 1×10\hat{ }9 J/cm². A dose of radiation or UV radiation may be measured in watt per square centimeter (W/cm²; fluence rate). A dose of radiation or UV radiation may be at least about 1×10\hat{ }−15 W/cm², 1×10\hat{ }−14 W/cm², 1×10\hat{ }−13 W/cm², 1×10\hat{ }−12 W/cm², 1×10\hat{ }−11 W/cm², 1×10\hat{ }−10 W/cm², 1×10\hat{ }−9 W/cm², 1×10\hat{ }−8 W/cm², 1×10\hat{ }−7 W/cm², 1×10\hat{ }−6 W/cm², 1×10\hat{ }−5 W/cm², 1×10\hat{ }−4 W/cm², 1×10\hat{ }−3 W/cm², 1×10\hat{ }−2 W/cm², 1×10\hat{ }−1 W/cm², 1×10\hat{ }0 W/cm², 1×10\hat{ }1 W/cm², 1×10\hat{ }2 W/cm², 1×10\hat{ }3 W/cm² 1×10\hat{ }4 W/cm², 1×10\hat{ }5 W/cm², 1×10\hat{ }6 W/cm², 1×10\hat{ }7 W/cm², 1×10\hat{ }8 W/cm², 1×10\hat{ }9 W/cm² or more. A dose of radiation or UV radiation may be at most about 1×10\hat{ }−15 W/cm², 1×10\hat{ }−14 W/cm², 1×10\hat{ }−13 W/cm², 1×10\hat{ }−12 W/cm², 1×10\hat{ }−11 W/cm², 1×10\hat{ }−10 W/cm², 1×10\hat{ }−9 W/cm², 1×10\hat{ }−8 W/cm², 1×10\hat{ }−7 W/cm², 1×10\hat{ }−6 W/cm², 1×10\hat{ }−5 W/cm², 1×10\hat{ }−4 W/cm², 1×10\hat{ }−3 W/cm², 1×10\hat{ }−2 W/cm², 1×10\hat{ }−1 W/cm², 1×10\hat{ }0 W/cm², 1×10\hat{ }1 W/cm², 1×10\hat{ }2 W/cm², 1×10\hat{ }4 W/cm², 1×10\hat{ }4 W/cm², 1×10\hat{ }5 W/cm², 1×10\hat{ }6 W/cm², 1×10\hat{ }7 W/cm², 1×10\hat{ }8 W/cm², or 1×10\hat{ }9 W/cm². Radiation or UV radiation (i.e., radiation stimulus or UV radiation stimulus) may be applied for at least about 1×10\hat{ }−9 seconds (s), 1×10\hat{ }−8 s, 1×10\hat{ }−7 s, 1×10\hat{ }−6 s, 1×10\hat{ }−5 s, 1×10\hat{ }−4 s, 1×10\hat{ }−3 s, 1×10\hat{ }−2 s, 1×10\hat{ }−1 s, 1×10\hat{ }0 s, 1×10\hat{ }1 s, 1×10\hat{ }2 s, 1×10\hat{ }3 s or more. The radiation or UV radiation (i.e., radiation stimulus or UV radiation stimulus) may be applied for at most about 1×10\hat{ }−9 seconds (s), 1×10\hat{ }−8 s, 1×10\hat{ }−7 s, 1×10\hat{ }−6 s, 1×10\hat{ }−5 s, 1×10\hat{ }−4 s, 1×10\hat{ }−3 s, 1×10\hat{ }−2 s, 1×10\hat{ }−1 s, 1×10\hat{ }0 s, 1×10\hat{ }1 s, 1×10\hat{ }2 s, 1×10\hat{ }3 s. The radiation stimulus or UV radiation stimulus may be applied to deliver a dose of UV as measured by fluence, using the formula: fluence=fluence rate×time (measured in seconds). The dose of the radiation or UV radiation or the strength of the radiation stimuli applied may be sufficient to cleave and/or excise any cleavable or excisable moieties described herein.

It will be appreciated that the cleaving agent may be used with a specific cleavable or excisable base. For example, excision of a DNA uracil base can be achieved by use of UDG, APE, Endonuclease VIII or USER. Similarly, an RNase can be used to excise an RNA base from a DNA backbone. Thus, different cleavable or excisable bases may be removed by different cleaving agents. This allows for simultaneous or sequential cleavage of cleavable or excisable bases. If at least two cleavable or excisable bases are cleaved by the same cleaving agent, then the at least two cleavable or excisable bases can be cleaved simultaneously. If a first cleavable base is cleaved by a first cleaving agent and a second cleavable base is cleaved by a second cleaving agent, then sequential cleaving can be done by first adding one cleaving agent and then the other. For example, if a nucleic acid molecule comprises a DNA uracil base and an RNA base, USER can be added first to remove the uracil and subsequently RNase can be added to remove the RNA base. Alternatively, the RNase could be added before the USER enzyme. The two cleaving agents could be added at the same step of a method or at different steps.

Sequencing of a first region (e.g., “seq1”) may be terminated before sequencing of a second region (e.g., “seq2”). In some cases, the termination can occur by incorporating a blocking moiety, such as a terminated nucleotide. In some cases, the terminated nucleotide is a dideoxynucleotide. In systems and methods where there are two primer molecules (e.g., as in FIGS. 8-9), and the first primer molecule is phosphorylated, the termination may comprise using a phosphate-dependent enzyme to degrade the first primer molecule. In systems and methods where a connected primer molecule is used, and the second primer region is phosphorylated, the termination may comprise using a phosphate-dependent enzyme to degrade the second primer region.

In some cases, prior to sequencing of a second region, a first extension strand extended from the first primer molecule may be degraded via an exonuclease. In some cases, such degradation may occur before activating the second primer molecule. Example exonucleases that can be used include the Lambda Exonuclease, T7 Exonuclease, and Exonuclease III. For example, in systems and methods where there are two primer molecules, where the first primer molecule comprises a 5′-phosphate and the second primer molecule does not have a 5′-phosphate, the first extension strand may be degraded using a Lambda Exonuclease. In another example, where the first primer molecule has an unprotected 5′ end and the second primer molecule has a protected 5′ end, the first extension strand may be degraded using a T7 Exonuclease. In another example, where a 3′ end of the second primer molecule is protected, the first extension strand may be degraded using an Exonuclease III. Where a connected primer molecule is used, and where a 3′ end of the first primer region is protected, the first extension strand may be degraded using an Exonuclease III.

The intermediary region disposed between the first target region and the second target region may comprise a sequence that is useful to be sequenced. The intermediary region may be of any length. For example, the intermediary region may be between 10 and 60 bases. In some cases, the intermediary region may be at least 1, 2, 3, 4, 5, 6, 7, 8, 9, 10, 20, 30, 40, 50, 60, 70, 80, 90, 100 or more bases in length. In some cases, the intermediary region may be at most 1, 2, 3, 4, 5, 6, 7, 8, 9, 10, 20, 30, 40, 50, 60, 70, 80, 90, 100 or more bases in length. In some cases, the intermediary region may comprise one or more functional sequences. In some cases, the intermediary region may comprise one or more (or multiples) of a barcode, sample index, and unique molecular identifier (UMI).

The first or second target region may comprise a polyT homopolymer region. As such, the first or second primer molecule may comprise a polyA sequence. In some cases, the polyA sequence may have a length of at least 1, 2, 3, 4, 5, 6, 7, 8, 9, 10, 20, 30, 40 or more bases. In some cases, the polyA sequence may have a length of at most 1, 2, 3, 4, 5, 6, 7, 8, 9, 10, 20, 30, 40 or more bases.

Also provided are compositions that comprise one or more reagents, supports, template nucleic acids, and/or intermediary sequencing complexes, prior to, during, and/or subsequent to one or more operations in the multi-priming sequencing workflows described herein.

Also provided are kits that comprise one or more reagents, supports, and/or template nucleic acids that can be used to perform one or more operations in the multi-priming sequencing workflows described herein. In some cases, a kit may comprise one or more machines configured to implement one or more operations, such as a light source configured to apply ultraviolet radiation to cleave a photocleavable base. Kits may include instructions for performing one or more operations in the multi-priming sequencing workflows described herein.

### Computer Systems

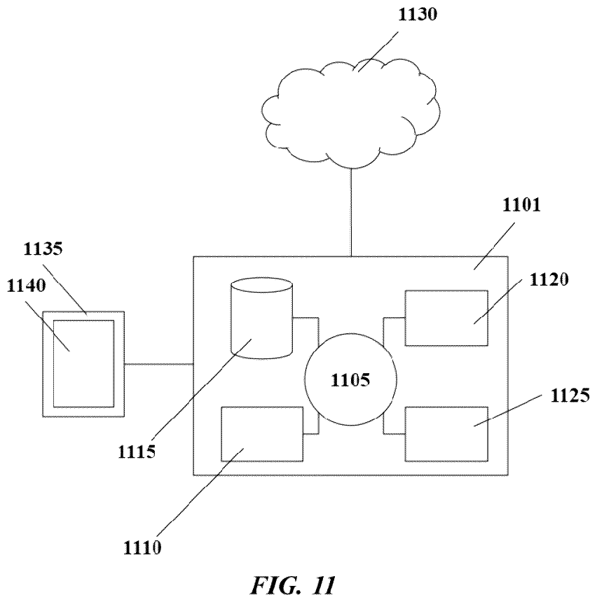

The present disclosure provides computer control systems that are programmed to implement methods of the disclosure. FIG. 11 shows a computer system 1101 that is programmed or otherwise configured to implement methods of the disclosure, such as to control the systems described herein (e.g., reagent dispensing, detecting, etc.) and collect, receive, and/or analyze sequencing information. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processor adapted to execute the code or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that comprises a user interface (UI) 1140 for providing, for example, provide—results of a nucleic acid sequence (e.g., sequencing signals, sequence reads, etc.). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105.

### NUMBERED EMBODIMENTS

### Embodiment 1

A method, comprising:
(a) hybridizing a first primer molecule and a second primer molecule to a first target region and a second target region, respectively, of a target nucleic acid strand, wherein an intermediary region is disposed between the first target region and the second target region on the target nucleic acid strand, wherein the second primer molecule is inactivated for extension;
(b) extending the first primer molecule to generate a first sequencing read for a first region of the target nucleic acid strand;
(c) activating the second primer molecule for extension; and
(d) extending the second primer molecule to generate a second sequencing read for a second region of the target nucleic acid strand, wherein the second region is different from the first region.

### Embodiment 2

The method of embodiment 1, wherein the target nucleic acid strand is coupled to a support.

### Embodiment 3

The method of embodiment 2, wherein the support is a bead.

### Embodiment 4

The method of embodiment 3, wherein the bead is immobilized to an individually addressable location of a plurality of individually addressable locations on a substrate.

### Embodiment 5

The method of embodiment 4, wherein the plurality of individually addressable locations comprises at least 1,000,000,000 individually addressable locations.

### Embodiment 6

The method of any one of embodiments 1-5, wherein the first target region is disposed 5′ to the second target region on the target nucleic acid strand.

### Embodiment 7

The method of any one of embodiments 1-5, wherein the first target region is disposed 3′ to the second target region on the target nucleic acid strand.

### Embodiment 8

The method of any one of embodiments 1-7, wherein the first region or the second region comprises at least a portion of the intermediary region.

### Embodiment 9

The method of any one of embodiments 1-8, wherein the second primer molecule is inactivated by a blocking moiety.

### Embodiment 10

The method of embodiment 9, wherein the blocking moiety comprises a dideoxynucleotide.

### Embodiment 11

The method of any one of embodiments 9-10, wherein the activating in (c) comprises cleaving the blocking moiety.

### Embodiment 12

The method of any one of embodiments 9-11, wherein the second primer molecule comprises one or more cleavage sites disposed 5′ of the blocking moiety.

### Embodiment 13

The method of embodiment 12, wherein the one or more cleavage sites comprises one or more members selected from the group consisting of: uracil, ribonucleotide, inosine, FapyG, 8oxoG, C3 spacer, photocleavable moiety, azobenzene, and abasic site.

### Embodiment 14

The method of embodiment 13, wherein the one or more cleavage sites comprises one or more uracils, and the activating in (c) comprises using a uracil-specific excision reagent (USER) enzyme to excise the one or more uracils.

### Embodiment 15

The method of embodiment 13, wherein the one or more cleavage sites comprises the photocleavable moiety, and the activating in (c) comprises applying ultraviolet radiation stimulus to the second primer molecule.

### Embodiment 16

The method of any one of embodiments 1-15, further comprising, prior to (d), terminating extension of the first primer molecule in (b).

### Embodiment 17

The method of embodiment 16, wherein the terminating occurs prior to (c).

### Embodiment 18

The method of any one of embodiments 16-17, wherein the terminating comprises incorporation of a terminated nucleotide.

### Embodiment 19

The method of embodiment 18, wherein the terminated nucleotide comprises a dideoxynucleotide.

### Embodiment 20

The method of any one of embodiments 16-17, wherein the first primer molecule is phosphorylated, and wherein the terminating comprises using a phosphate-dependent enzyme to degrade the first primer molecule.

### Embodiment 21

The method of any one of embodiments 1-20, further comprising, prior to (d), degrading a first extension strand extended from the first primer molecule via an exonuclease.

### Embodiment 22

The method of embodiment 21, wherein the degrading occurs prior to (c).

### Embodiment 23

The method of any one of embodiments 21-22, wherein the first primer molecule comprises a 5′-phosphate, wherein the second primer molecule does not have a 5′-phosphate, and wherein the first extension strand is degraded using a Lambda Exonuclease.

### Embodiment 24

The method of any one of embodiments 21-22, wherein a 5′ end of the second primer molecule is protected, wherein a 5′ end of the first primer molecule is unprotected, and wherein the first extension strand is degraded using a T7 Exonuclease.

### Embodiment 25

The method of any one of embodiments 21-22, wherein a 3′ end of the second primer molecule is protected, and wherein the first extension strand is degraded using an Exonuclease III.

### Embodiment 26

The method of any one of embodiments 1-25, wherein the intermediary region comprises one or more of a barcode, a sample index, and a unique molecular identifier.

### Embodiment 27

The method of any one of embodiments 1-26, wherein the intermediary region comprises between 10 and 60 bases.

### Embodiment 28

The method of any one of embodiments 1-27, wherein the first target region or the second target region comprises a polyT homopolymer region.

### Embodiment 29

The method of embodiment 28, wherein the first primer molecule or the second primer molecule comprises a polyA sequence of at least 10 bases in length.

### Embodiment 30

The method of any one of embodiments 1-29, further comprising associating the first sequencing read and the second sequencing read via a respective individually addressable location of sequencing signals detected on a substrate during generation of the first sequencing read and the second sequencing read.

### Embodiment 31

A composition, comprising:
a target nucleic acid strand comprising a first target region and a second target region, wherein an intermediary region is disposed between the first target region and the second target region on the target nucleic acid strand;
a first primer molecule comprising a first sequence complementary to the first target region; and
a second primer molecule comprising a second sequence complementary to the second target region, wherein the second primer molecule is inactivated for extension.

### Embodiment 32

The composition of embodiment 31, further comprising a support coupled to the target nucleic acid strand.

### Embodiment 33

The composition of embodiment 32, wherein the support is a bead.

### Embodiment 34

The composition of embodiment 33, wherein the bead is immobilized to an individually addressable location of a plurality of individually addressable locations on a substrate.

### Embodiment 35

The composition of embodiment 34, wherein the plurality of individually addressable locations comprises at least 1,000,000,000 individually addressable locations.

### Embodiment 36

The composition of any one of embodiments 31-35, wherein the first target region is disposed 5′ to the second target region on the target nucleic acid strand.

### Embodiment 37

The composition of any one of embodiments 31-35, wherein the first target region is disposed 3′ to the second target region on the target nucleic acid strand.

### Embodiment 38

The composition of any one of embodiments 31-37, wherein the first primer molecule or the second primer molecule is hybridized to the target nucleic acid strand.

### Embodiment 39

The composition of embodiment 38, wherein the first primer molecule and the second primer molecule are hybridized to the target nucleic acid strand.

### Embodiment 40

The composition of embodiment 39, further comprising a growing strand extended from the first primer molecule and hybridized to the target nucleic acid strand.

### Embodiment 41

The composition of embodiment 40, wherein the growing strand comprises a terminated nucleotide at a 3′ end.

### Embodiment 42

The composition of embodiment 41, wherein the terminated nucleotide comprises a dideoxynucleotide.

### Embodiment 43

The composition of any one of embodiments 40-42, further comprising an exonuclease.

### Embodiment 44

The composition of embodiment 43, wherein the first primer molecule comprises a 5′-phosphate, wherein the second primer molecule does not have a 5′-phosphate, and wherein the exonuclease is a Lambda Exonuclease.

### Embodiment 45

The composition of embodiment 43, wherein a 5′ end of the second primer molecule is protected, wherein a 5′ end of the first primer molecule is unprotected, and wherein the exonuclease is a T7 Exonuclease.

### Embodiment 46

The composition of embodiment 43, wherein a 3′ end of the second primer molecule is protected, and wherein the exonuclease is an Exonuclease III.

### Embodiment 47

The composition of any one of embodiments 31-46, wherein the second primer molecule is inactivated for extension by a blocking moiety.

### Embodiment 48

The composition of embodiment 47, wherein the blocking moiety comprises a dideoxynucleotide.

### Embodiment 49

The composition of any one of embodiments 47-48, wherein the second primer molecule comprises one or more cleavage sites disposed 5′ of the blocking moiety.

### Embodiment 50

The composition of embodiment 49, wherein the one or more cleavage sites comprises one or more members selected from the group consisting of uracil, ribonucleotide, inosine, FapyG, 8oxoG, C3 spacer, photocleavable moiety, azobenzene, and abasic site.

### Embodiment 51

The composition of embodiment 50, wherein the one or more cleavage sites comprises one or more uracils.

### Embodiment 52

The composition of embodiment 50, wherein the one or more cleavage sites comprises the photocleavable moiety.

### Embodiment 53

The composition of any one of embodiments 49-52, further comprising a cleavage reagent configured to cleave the second primer molecule at the one or more cleavage sites.

### Embodiment 54

The composition of embodiment 53, wherein the cleavage reagent comprises a uracil-specific excision reagent (USER) enzyme.

### Embodiment 55

The composition of embodiment 53, wherein the cleavage reagent comprises a dose of ultraviolet radiation.

### Embodiment 56

The composition of any one of embodiments 31-55, further comprising an extension termination reagent configured to terminate an extension of the first primer molecule.

### Embodiment 57

The composition of embodiment 56, wherein the first primer molecule is phosphorylated, and wherein the extension termination reagent comprises a phosphate-dependent enzyme.

### Embodiment 58

The composition of any one of embodiments 31-57, wherein the intermediary region comprises one or more of a barcode, a sample index, and a unique molecular identifier.

### Embodiment 59

The composition of any one of embodiments 31-58, wherein the intermediary region comprises between 10 and 60 bases.

### Embodiment 60

The composition of any one of embodiments 31-59, wherein the first target region or the second target region comprises a polyT homopolymer region.

### Embodiment 61

The composition of embodiment 60, wherein the first primer molecule or the second primer molecule comprises a polyA sequence of at least 10 bases in length.

### Embodiment 62

A kit, comprising:
a first primer molecule comprising a first sequence complementary to a first target region;
a second primer molecule comprising a second sequence complementary to a second target region, wherein the second primer molecule is inactivated for extension; and
an activation reagent configured to activate the second primer molecule for extension, or instructions to activate the second primer molecule for extension.

### Embodiment 63

The kit of embodiment 62, further comprising a termination reagent configured to terminate an extension reaction of the first primer molecule.

### Embodiment 64

The kit of embodiment 63, wherein the termination reagent comprises a dideoxynucleotide.

### Embodiment 65

The kit of any one of embodiments 63-64, wherein the first primer molecule is phosphorylated, and wherein the termination reagent comprises a phosphate-dependent enzyme.

### Embodiment 66

The kit of any one of embodiments 62-65, further comprising a degradation reagent configured to degrade a first extension strand extended from the first primer molecule.

### Embodiment 67

The kit of embodiment 66, wherein the degradation reagent is an exonuclease.

### Embodiment 68

The kit of embodiment 67, wherein the first primer molecule comprises a 5′-phosphate, wherein the second primer molecule does not have a 5′-phosphate, and wherein the exonuclease is a Lambda Exonuclease.

### Embodiment 69

The kit of embodiment 67, wherein a 5′ end of the second primer molecule is protected, wherein a 5′ end of the first primer molecule is unprotected, and wherein the exonuclease is a T7 Exonuclease.

### Embodiment 70

The kit of embodiment 67, wherein a 3′ end of the second primer molecule is protected, and the exonuclease is an Exonuclease III.

### Embodiment 71

The kit of any one of embodiments 62-70, wherein the second primer molecule is inactivated for extension by a blocking moiety.

### Embodiment 72

The kit of embodiment 71, wherein the blocking moiety comprises a dideoxynucleotide.

### Embodiment 73

The kit of any one of embodiments 71-72, wherein the second primer molecule comprises one or more cleavage sites disposed 5′ of the blocking moiety.

### Embodiment 74

The kit of embodiment 73, wherein the one or more cleavage sites comprises one or more members selected from the group consisting of: uracil, ribonucleotide, inosine, FapyG, 8oxoG, C3 spacer, photocleavable moiety, azobenzene, and abasic site.

### Embodiment 75

The kit of embodiment 74, wherein the one or more cleavage sites comprises one or more uracils.

### Embodiment 76

The kit of embodiment 74, wherein the one or more cleavage sites comprises the photocleavable moiety.

### Embodiment 77

The kit of any one of embodiments 73-76, wherein the activation reagent comprises a cleavage reagent configured to cleave the second primer molecule at the one or more cleavage sites.

### Embodiment 78

The kit of embodiment 77, wherein the cleavage reagent comprises a uracil-specific excision reagent (USER) enzyme.

### Embodiment 79

The kit of embodiment 77, wherein the cleavage reagent comprises a dose of ultraviolet radiation or the instructions comprise instructions to apply the dose of ultraviolet radiation at a predetermined wavelength.

### Embodiment 80

The kit of any one of embodiments 62-79, wherein the first target region or the second target region comprises a polyT homopolymer region.

### Embodiment 81

The kit of any one of embodiments 62-80, wherein the first primer molecule or the second primer molecule comprises a polyA sequence of at least 10 bases in length.

### Embodiment 82

A method, comprising:
(a) hybridizing a first primer region and a second primer region of a primer molecule to a first target region and a second target region, respectively, of a target nucleic acid strand, wherein an intermediary region is disposed between the first target region and the second target region on the target nucleic acid strand, wherein a connector region is disposed between the first primer region and the second primer region;
(b) extending the primer molecule from the second primer region to generate a first sequencing read for a first region of the target nucleic acid strand;
(c) cleaving the primer molecule to activate the first primer region for extension; and
(d) extending the primer molecule from the first primer region to generate a second sequencing read for a second region of the target nucleic acid strand, wherein the second region is different from the first region.

### Embodiment 83

The method of embodiment 82, wherein the target nucleic acid strand is coupled to a support.

### Embodiment 84

The method of embodiment 83, wherein the support is a bead.

### Embodiment 85

The method of embodiment 84, wherein the bead is immobilized to an individually addressable location of a plurality of individually addressable locations on a substrate.

### Embodiment 86

The method of embodiment 85, wherein the plurality of individually addressable locations comprises at least 1,000,000,000 individually addressable locations.

### Embodiment 87

The method of any one of embodiments 82-86, wherein the first target region is disposed 3′ to the second target region on the target nucleic acid strand.

### Embodiment 88

The method of any one of embodiments 82-87, wherein the first region or the second region comprises at least a portion of the intermediary region.

### Embodiment 89

The method of any one of embodiments 82-88, wherein the primer molecule comprises one or more cleavage sites disposed between the first primer region and the connector region.

### Embodiment 90

The method of embodiment 89, wherein the one or more cleavage sites comprises one or more members selected from the group consisting of: uracil, ribonucleotide, inosine, FapyG, 8oxoG, C3 spacer, photocleavable moiety, azobenzene, and abasic site.

### Embodiment 91

The method of embodiment 90, wherein the one or more cleavage sites comprises one or more uracils, and (c) comprises using a uracil-specific excision reagent (USER) enzyme to excise the one or more uracils.

### Embodiment 92

The method of embodiment 90, wherein the one or more cleavage sites comprises the photocleavable moiety, and (c) comprises applying ultraviolet radiation stimulus to the primer molecule.

### Embodiment 93

The method of any one of embodiments 82-92, further comprising, prior to (d), terminating extension of the second primer region in (b).

### Embodiment 94

The method of embodiment 93, wherein the terminating occurs prior to (c).

### Embodiment 95

The method of any one of embodiments 93-94, wherein the terminating comprises incorporation of a terminated nucleotide.

### Embodiment 96

The method of embodiment 95, wherein the terminated nucleotide comprises a dideoxynucleotide.

### Embodiment 97

The method of any one of embodiments 95-96, wherein the second primer region is phosphorylated, and the terminating comprises using a phosphate-dependent enzyme to degrade the second primer region.

### Embodiment 98

The method of any one of embodiments 82-97, further comprising, prior to (d), degrading a first extension strand extended from the second primer region via an exonuclease.

### Embodiment 99

The method of embodiment 98, wherein a 3′ end of the first primer region is protected, and the first extension strand is degraded using an Exonuclease III.

### Embodiment 100

The method of any one of embodiments 82-99, wherein the intermediary region comprises one or more of a barcode, a sample index, and a unique molecular identifier.

### Embodiment 101

The method of any one of embodiments 82-100, wherein the intermediary region comprises between 10 and 60 bases.

### Embodiment 102

The method of any one of embodiments 82-101, wherein the first target region or the second target region comprises a polyT homopolymer region.

### Embodiment 103

The method of embodiment 102, wherein the first primer region or the second primer region comprises a polyA sequence of at least 10 bases in length.

### Embodiment 104

The method of any one of embodiments 82-103, further comprising associating the first sequencing read and the second sequencing read via a respective individually addressable location of sequencing signals detected on a substrate during generation of the first sequencing read and the second sequencing read.

### Embodiment 105

A composition, comprising:
a target nucleic acid strand comprising a first target region and a second target region, wherein an intermediary region is disposed between the first target region and the second target region on the target nucleic acid strand; and
a primer molecule comprising a first primer region complementary to the first target region and a second primer region complementary to the second target region, wherein a connector region is disposed between the first primer region and the second primer region, and wherein one or more cleavage sites are disposed between the first primer region and the connector region.

### Embodiment 106

The composition of embodiment 105, further comprising a support coupled to the target nucleic acid strand.

### Embodiment 107

The composition of embodiment 106, wherein the support is a bead.

### Embodiment 108

The composition of embodiment 107, wherein the bead is immobilized to an individually addressable location of a plurality of individually addressable locations on a substrate.

### Embodiment 109

The composition of embodiment 108, wherein the plurality of individually addressable locations comprises at least 1,000,000,000 individually addressable locations.

### Embodiment 110

The composition of any one of embodiments 105-109, wherein the first target region is disposed 3′ to the second target region on the target nucleic acid strand.

### Embodiment 111

The composition of any one of embodiments 105-110, wherein the first primer region or the second primer region is hybridized to the target nucleic acid strand.

### Embodiment 112

The composition of embodiment 111, wherein the first primer region and the second primer region are hybridized to the target nucleic acid strand.

### Embodiment 113

The composition of embodiment 112, further comprising a growing strand extended from the second primer region and hybridized to the target nucleic acid strand.

### Embodiment 114

The composition of embodiment 113, wherein the growing strand comprises a terminated nucleotide at a 3′ end.

### Embodiment 115

The composition of embodiment 114, wherein the terminated nucleotide comprises a dideoxynucleotide.

### Embodiment 116

The composition of any one of embodiments 112-114, further comprising an exonuclease.

### Embodiment 117

The composition of embodiment 116, wherein a 3′ end of the first primer region is protected, and wherein the exonuclease is an Exonuclease III.

### Embodiment 118

The composition of any one of embodiments 105-117, wherein the one or more cleavage sites comprises one or more members selected from the group consisting of: uracil, ribonucleotide, inosine, FapyG, 8oxoG, C3 spacer, photocleavable moiety, azobenzene, and abasic site.

### Embodiment 119

The composition of embodiment 118, wherein the one or more cleavage sites comprises one or more uracils.

### Embodiment 120

The composition of embodiment 118, wherein the one or more cleavage sites comprises the photocleavable moiety.

### Embodiment 121

The composition of any one of embodiments 105-120, further comprising a cleavage reagent configured to cleave the primer molecule at the one or more cleavage sites.

### Embodiment 122

The composition of embodiment 121, wherein the cleavage reagent comprises a uracil-specific excision reagent (USER) enzyme.

### Embodiment 123

The composition of embodiment 121, wherein the cleavage reagent comprises a dose of ultraviolet radiation.

### Embodiment 124

The composition of any one of embodiments 105-123, further comprising an extension termination reagent configured to terminate an extension of the second primer region.

### Embodiment 125

The composition of embodiment 124, wherein the second primer region is phosphorylated, and wherein the extension termination reagent comprises a phosphate-dependent enzyme.

### Embodiment 126

The composition of embodiment 124, wherein the extension termination reagent comprises a dideoxynucleotide.

### Embodiment 127

The composition of any one of embodiments 105-126, wherein the intermediary region comprises one or more of a barcode, a sample index, and a unique molecular identifier.

### Embodiment 128

The composition of any one of embodiments 105-127, wherein the intermediary region comprises between 10 and 60 bases.

### Embodiment 129

The composition of any one of embodiments 105-128, wherein the first target region or the second target region comprises a polyT homopolymer region.

### Embodiment 130

The composition of embodiment 129, wherein the first primer region or the second primer region comprises a polyA sequence of at least 10 bases in length.

### Embodiment 131

A kit, comprising:
a primer molecule comprising, in a 5′ to 3′ direction, a first primer region complementary to a first target region, a connector region, and a second primer region complementary to a second target region, wherein one or more cleavage sites are disposed between the first primer region and the connector region; and
an activation reagent configured to activate the first primer region for extension, or instructions to activate the first primer region for extension.

### Embodiment 132

The kit of embodiment 131, further comprising a termination reagent configured to terminate an extension reaction of the second primer region.

### Embodiment 133

The kit of embodiment 132, wherein the termination reagent comprises a dideoxynucleotide.

### Embodiment 134

The kit of any one of embodiments 131-132, wherein the second primer region is phosphorylated, and wherein the termination reagent comprises a phosphate-dependent enzyme.

### Embodiment 135

The kit of any one of embodiments 131-134, further comprising a degradation reagent configured to degrade a first extension strand extended from the second primer region.

### Embodiment 136

The kit of embodiment 135, wherein the degradation reagent is an exonuclease.

### Embodiment 137

The kit of embodiment 136, wherein a 3′ end of the first primer region is protected, and wherein the exonuclease is an Exonuclease III.

### Embodiment 138

The kit of any one of embodiments 131-137, wherein the one or more cleavage sites comprises one or more members selected from the group consisting of: uracil, ribonucleotide, inosine, FapyG, 8oxoG, C3 spacer, photocleavable moiety, azobenzene, and abasic site.

### Embodiment 139

The kit of embodiment 138, wherein the one or more cleavage sites comprises one or more uracils.

### Embodiment 140

The kit of any one of embodiments 131-139, wherein the activation reagent comprises a cleavage reagent configured to cleave the primer molecule at the one or more cleavage sites.

### Embodiment 141

The kit of embodiment 140, wherein the cleavage reagent comprises a uracil-specific excision reagent (USER) enzyme.

### Embodiment 142

The kit of embodiment 140, wherein the cleavage reagent comprises a dose of ultraviolet radiation or the instructions comprise instructions to apply the dose of ultraviolet radiation at a predetermined wavelength.

### Embodiment 143

The kit of any one of embodiments 131-142, wherein the first target region or the second target region comprises a polyT homopolymer region.

### Embodiment 144

The kit of any one of embodiments 131-143, wherein the first primer region or the second primer region comprises a polyA sequence of at least 10 bases in length.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-144. (canceled)

145. A method, comprising:
(a) hybridizing a first primer region and a second primer region of a primer molecule to a first target region and a second target region, respectively, of a target nucleic acid strand, wherein an intermediary region is disposed between the first target region and the second target region on the target nucleic acid strand, wherein a connector region is disposed between the first primer region and the second primer region;
(b) extending the primer molecule from the second primer region to generate a first sequencing read for a first region of the target nucleic acid strand;
(c) cleaving the primer molecule to activate the first primer region for extension; and
(d) extending the primer molecule from the first primer region to generate a second sequencing read for a second region of the target nucleic acid strand, wherein the second region is different from the first region.

146. The method of claim 145, wherein the target nucleic acid strand is coupled to a support.

147. The method of claim 146, wherein the support is a bead, and wherein the bead is immobilized to an individually addressable location of a plurality of individually addressable locations on a substrate.

148. The method of claim 147, wherein the plurality of individually addressable locations comprises at least 1,000,000,000 individually addressable locations.

149. The method of claim 145, wherein the first target region is disposed 3′ to the second target region on the target nucleic acid strand.

150. The method of claim 145, wherein the first region or the second region comprises at least a portion of the intermediary region.

151. The method of claim 145, wherein the primer molecule comprises one or more cleavage sites disposed between the first primer region and the connector region.

152. The method of claim 151, wherein the one or more cleavage sites comprises one or more members selected from the group consisting of: uracil, ribonucleotide, inosine, FapyG, 8oxoG, C3 spacer, photocleavable moiety, azobenzene, and abasic site.

153. The method of claim 152, wherein the one or more cleavage sites comprises one or more uracils, and (c) comprises using a uracil-specific excision reagent (USER) enzyme to excise the one or more uracils.

154. The method of claim 145, further comprising, prior to (d), terminating extension of the second primer region in (b).

155. The method of claim 154, wherein the terminating occurs prior to (c).

156. The method of claim 154, wherein the terminating comprises incorporation of a terminated nucleotide.

157. The method of claim 156, wherein the second primer region is phosphorylated, and the terminating comprises using a phosphate-dependent enzyme to degrade the second primer region.

158. The method of claim 145, further comprising, prior to (d), degrading a first extension strand extended from the second primer region via an exonuclease.

159. The method of claim 158, wherein a 3′ end of the first primer region is protected, and the first extension strand is degraded using an Exonuclease III.

160. The method of claim 145, wherein the intermediary region comprises one or more of a barcode, a sample index, and a unique molecular identifier.

161. The method of claim 145, wherein the intermediary region comprises between 10 and 60 bases.

162. The method of claim 145, wherein the first target region or the second target region comprises a polyT homopolymer region.

163. The method of claim 162, wherein the first primer region or the second primer region comprises a polyA sequence of at least 10 bases in length.

164. The method of claim 145, further comprising associating the first sequencing read and the second sequencing read via a respective individually addressable location of sequencing signals detected on a substrate during generation of the first sequencing read and the second sequencing read.
